SEQUENCE LISTING (32 sequences)

SEQ ID NO: 1 - DNA, 20 bp, Artificial
  An artificially synthesized primer sequence for RT-PCR
cgaccactttgtcaagctca

SEQ ID NO: 2 - DNA, 23 bp, Artificial
  An artificially synthesized primer sequence for RT-PCR
ggttgagcacagggtactttatt

SEQ ID NO: 3 - DNA, 24 bp, Artificial
  An artificially synthesized primer sequence for RT-PCR
gagtccaggtaagtgaatctgtcc

SEQ ID NO: 4 - DNA, 22 bp, Artificial
  An artificially synthesized primer sequence for RT-PCR
atttccaccgagacctctcatc

SEQ ID NO: 5 - DNA, 25 bp, Artificial
  An artificially synthesized primer sequence for RT-PCR
cggaattcatgaggctcctccgcag

SEQ ID NO: 6 - DNA, 30 bp, Artificial
  An artificially synthesized primer sequence for RT-PCR
ccgctcgaggacaaagagccacaactgatg

SEQ ID NO: 7 - DNA, 39 bp, Artificial
  An artificially synthesized primer sequence for RT-PCR
gctcacgttcctggatgccgactgtgagtgcttccacgg

SEQ ID NO: 8 - DNA, 39 bp, Artificial
  An artificially synthesized primer sequence for RT-PCR
ccgtggaagcactcacagtcggcatccaggaacgtgagc

SEQ ID NO: 9 - DNA, 42 bp, Artificial
  An artificially synthesized primer sequence for RT-PCR
cagatggagatctggggagggcagaacgtggaaatgtccttc

SEQ ID NO: 10 - DNA, 42 bp, Artificial
  An artificially synthesized primer sequence for RT-PCR
gaaggacatttccacgttctgccctccccagatctccatctg

SEQ ID NO: 11 - DNA, 29 bp, Artificial
  An artificially synthesized primer sequence for RT-PCR
ccggaattcgaggaggccacagagaagcc

SEQ ID NO: 12 - DNA, 29 bp, Artificial
  An artificially synthesized primer sequence for RT-PCR
ccgctcgagggtggtggccagtggggggc

SEQ ID NO: 13 - DNA, 37 bp, Artificial
  An artificially synthesized primer sequence for PCR
ataagaatgcggccgcagaggaggccacagagaagcc

SEQ ID NO: 14 - DNA, 30 bp, Artificial
  An artificially synthesized primer sequence for PCR
cgcggatccgacaaagagccacaactgatg

SEQ ID NO: 15 - protein, 20 aa, Artificial
  An artificially signal sequence peptide
METDTLLLWVLLLWVPGSTG

SEQ ID NO: 16 - RNA, 19 nt, Artificial Sequence
  An artificially synthesized sequence for siRNA
gcagcacgacuucuucaag

SEQ ID NO: 17 - RNA, 19 nt, Artificial Sequence
  An artificially synthesized sequence for siRNA
gagaaauccuucggugaca

SEQ ID NO: 18 - RNA, 19 nt, Artificial Sequence
  An artificially synthesized  sequence for siRNA
guucagugcccagcucuac

SEQ ID NO: 19 - DNA, 26 bp, Artificial
  An artificially synthesized  sequence for siRNA
caccgtgtcttcaagcttgaagacta

SEQ ID NO: 20 - DNA, 26 bp, Artificial
  An artificially synthesized  sequence for siRNA
aaaatagtcttcaagcttgaagacac

SEQ ID NO: 21 - DNA, 19 bp, Artificial
  An artificially synthesized target sequence for shRNA
gaagcagcacgacttcttc

SEQ ID NO: 22 - DNA, 19 bp, Artificial
  An artificially synthesized target sequence for shRNA
gcactgtttcaatgccttt

SEQ ID NO: 23 - DNA, 19 bp, Artificial
  An artificially synthesized target sequence for shRNA
gagaaatccttcggtgaca

SEQ ID NO: 24 - DNA, 19 bp, Artificial
  An artificially synthesized  sequence for siRNA
cagaattccatcggtgact

SEQ ID NO: 25 - DNA, 19 bp, Artificial
  An artificially synthesized  sequence for siRNA
cagaactccatcggtgact

SEQ ID NO: 26 - protein, 11 aa, Artificial
  An artificially polypeptide
AHGVTSAPDTR

SEQ ID NO: 27 - protein, 11 aa, Artificial
  An artificially polypeptide
RPAPGSTAPPA

SEQ ID NO: 28 - DNA, 4520 bp, Homo sapiens
ccaggctggtccctcctgccttcgctctctgctggtcgcggtggcctgtagctcctccctcggtaggacccccctccaccttggcgcttacgagatgaatgaggagcccagacctctggaacttggagggttgttcagattcccaggctgacaaaaaggagaaacagcttttacttgcatccagggccaatcatcgcagacccagacgtctgcagagggctgaggccccagcttgtggccaccacaacgtatcaagctatctccagggttgggctcaggactcagagctgacgcagctggggtgccccttggttctggaggatgaggctcctccgcagacgccacatgcccctgcgcctggccatggtgggctgcgcctttgtgctcttcctcttcctcctgcatagggatgtgagcagcagagaggaggccacagagaagccgtggctgaagtccctggtgagccggaaggatcacgtcctggacctcatgctggaggccatgaacaaccttagagattcaatgcccaagctccaaatcagggctccagaagcccagcagactctgttctccataaaccagtcctgcctccctgggttctataccccagctgaactgaagcccttctgggaacggccaccacaggaccccaatgcccctggggcagatggaaaagcatttcagaagagcaagtggacccccctggagacccaggaaaaggaagaaggctataagaagcactgtttcaatgcctttgccagcgaccggatctccctgcagaggtccctggggccagacacccgaccacctgagtgtgtggaccagaagttccggcgctgccccccactggccaccaccagcgtgatcattgtgttccacaacgaagcctggtccacactgctgcgaacagtgtacagcgtcctacacaccacccctgccatcttgctcaaggagatcatactggtggatgatgccagcacagaggagcacctaaaggagaagctggagcagtacgtgaagcagctgcaggtggtgagggtggtgcggcaggaggagcggaaggggctgatcaccgcccggctgctgggggccagcgtggcacaggcggaggtgctcacgttcctggatgcccactgtgagtgcttccacggctggctggagcccctcctggctcgaatcgctgaggacaagacagtggtggtgagcccagacatcgtcaccatcgaccttaatacttttgagttcgccaagcccgtccagaggggcagagtccatagccgaggcaactttgactggagcctgaccttcggctgggaaacacttcctccacatgagaagcagaggcgcaaggatgaaacctaccccatcaaatccccgacgtttgctggtggcctcttctccatctccaagtcctactttgagcacatcggtacctatgataatcagatggagatctggggaggggagaacgtggaaatgtccttccgggtgtggcagtgtgggggccagctggagatcatcccctgctctgtcgtaggccatgtgttccggaccaagagcccccacaccttccccaagggcactagtgtcattgctcgcaatcaagtgcgcctggcagaggtctggatggacagctacaagaagattttctataggagaaatctgcaggcagcaaagatggcccaagagaaatccttcggtgacatttcggaacgactgcagctgagggaacaactgcactgtcacaacttttcctggtacctgcacaatgtctacccagagatgtttgttcctgacctgacgcccaccttctatggtgccatcaagaacctcggcaccaaccaatgcctggatgtgggtgagaacaaccgcggggggaagcccctcatcatgtactcctgccacggccttggcggcaaccagtactttgagtacacaactcagagggaccttcgccacaacatcgcaaagcagctgtgtctacatgtcagcaagggtgctctgggccttgggagctgtcacttcactggcaagaatagccaggtccccaaggacgaggaatgggaattggcccaggatcagctcatcaggaactcaggatctggtacctgcctgacatcccaggacaaaaagccagccatggccccctgcaatcccagtgacccccatcagttgtggctctttgtctaggacccagatcatccccagagagagcccccacaagctcctcaggaaacaggattgctgatgtctgggaacctgatcaccagcttctctggaggccgtaaagatggatttctaaacccactgggtggcaaggcaggaccttcctaatccttgcaacaacattgggcccattttctttccttcacaccgatggaagagaccattaggacatatatttagcctagcgttttcctgttctagaaatagaggctcccaaagtagggaaggcagctgggggagggttcagggcagcaatgctgagttcaagaaaagtacttcaggctgggcacagtggctcatgcctgaaatcctagcactttgggaagacaatgtgggagaatggcttgagcccaggagttcaagaccggcctgagcaacatagtgaggatcccatctctacgcccaccctccccccggcaaaaaaaaaaagctgggtatggtggcttatgcctgtagtcgcagctactcagaaggctgaggtgggaggattgcttgttccccggaggttgaagctacagtgagccttgattgtgtcactgcactccagcctgggcaacaggtaagactctgtctcaaaaaaaaacaaaaaagaagaagaaaagtacttctacagccatgtcctattccttgatcatccaaagcacctgcagagtccagtgaaatgatatattctggctgggcacagtggctcacacctgtaatcctagcactttgggaggccaaggcaggtggatcacctgaggtcagaagtttgaaaccagcctggactacatggtgaaactccatctctactaaaagtacaaaaattagctgggcatgatggcacgcacctgcagtcccagctacttgggaggctgaggcaggagaatcactcgaacccaggaggcagaggttgcagtgagccaagacagcaccattgcaccccagcctgagcaacaagagcgaaactccatctcaggaaaaaaaaaaaaaaaaaaagtatattctaacagacagatcagaggtctaagagatcctcccttgctattattacctgaagtctgtagaactgtttacagatatctccttgacaggtgtcctttatcttactttatctgtacagtaatcctgtgagaaagacaggacagaaaccactgtgcctattttacagatacgaaaactgagacacaggtaaaggggcttgtctgtagtcccatagctagcagatggctggagccaagactgaggctcgttcttcaatgctgagccagggctccttccgctgcaccacaagaacgctagaccactcgccaccagccttctcattccctcttcctccattctaatcatttctagctggctggcctccacagagcataggaaaacagccagggccgggcacggtggctcatgcctgtaatctcaacactctgggaggccgagccgggtggataacctgaggtcaggaattcgagaccagcctggccaacatggtaaaaccccatctctactaaaaatataaaaattagccaggcatggtggcgcacacctgtaatcccagctactcaagaggctgaggcaggagaattgcttaaatctgggaggcggaagttgcagtgagccaagatcgcgccactgaactccagcctaggcaacaagagcaaaactccatctccaaaaaaaagaaaggaaaaacagggccaggtagccattgtggagagagcacacttaggaatcctgggatgttagtgttaaaagaaagctcctggagccagtgattctcaggtttgtcccagaacccttttttctaagccccatataaaaggtagattaaaaaaacaaagtagcatgagtgaaattgagagagggacaggtaatgccttccagcccctaacttctaacaatctggaagcacaacgtgaaaatcacgtagcccaaccctatcattttcatattatgaaactgagtccaggtaagtgaatctgtccaaggtcacccagcaaggtatcagtagccctgagggtaaggactctgataaggctcgggagggtcctggaaagcctgaggcggcaggaagagtgtgcagagttgagcgtgtctggaaggctgatccactgctgggcccacatcaaagcccccatggggagcagacccgactgcacatggctcttttgctggaagaagagcatggctgcgcagaggactaaaatttcatctgggaaggcttcttttgactgtcagtagcaggatgtcaccagatgagggtgctatgggaccacagctgtctttgttcccattgcaactcaaccctgcgg

SEQ ID NO: 29 - protein, 622 aa, Homo sapiens
MRLLRRRHMPLRLAMVGCAFVLFLFLLHRDVSSREEATEKPWLKSLVSRKDHVLDLMLEAMNNLRDSMPKLQIRAPEAQQTLFSINQSCLPGFYTPAELKPFWERPPQDPNAPGADGKAFQKSKWTPLETQEKEEGYKKHCFNAFASDRISLQRSLGPDTRPPECVDQKFRRCPPLATTSVIIVFHNEAWSTLLRTVYSVLHTTPAILLKEIILVDDASTEEHLKEKLEQYVKQLQVVRVVRQEERKGLITARLLGASVAQAEVLTFLDAHCECFHGWLEPLLARIAEDKTVVVSPDIVTIDLNTFEFAKPVQRGRVHSRGNFDWSLTFGWETLPPHEKQRRKDETYPIKSPTFAGGLFSISKSYFEHIGTYDNQMEIWGGENVEMSFRVWQCGGQLEIIPCSVVGHVFRTKSPHTFPKGTSVIARNQVRLAEVWMDSYKKIFYRRNLQAAKMAQEKSFGDISERLQLREQLHCHNFSWYLHNVYPEMFVPDLTPTFYGAIKNLGTNQCLDVGENNRGGKPLIMYSCHGLGGNQYFEYTTQRDLRHNIAKQLCLHVSKGALGLGSCHFTGKNSQVPKDEEWELAQDQLIRNSGSGTCLTSQDKKPAMAPCNPSDPHQLWLFV

SEQ ID NO: 30 - DNA, 1209 bp, Homo sapiens
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaa

SEQ ID NO: 31 - protein, 273 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 32 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

(19) United States
(12) Patent Application Publication
(10) Pub. No.: US 2012/0214174 A1
(43) Pub. Date: Aug. 23, 2012

(54) GENE AND POLYPEPTIDE RELATING TO BREAST CANCER

(75) Inventors: Toyomasa Katagiri, Tokyo (JP); Yusuke Nakamura, Tokyo (JP); Takuya Tsunoda, Kanagawa (JP)

(73) Assignee: OncoTherapy Science, Inc., Kanagawa (JP)

(21) Appl. No.: 13/392,058

(22) PCT Filed: Aug. 19, 2010
(86) PCT No.: PCT/JP2010/005115

§ 371 Date: Apr. 27, 2012

Related U.S. Application Data
(60) Provisional application No. 61/275,197, filed on Aug. 25, 2009.

(51) Int. Cl. G01N 33/566; C12Q 1/48

(52) U.S. Cl. 435/7.23; 435/7.1; 435/15

(57) ABSTRACT

Herein disclosed are methods of identifying substances suitable for the treatment and prevention of cancer, particularly cancers associated with the overexpression of GALNT6 gene. Methods of the present invention use or target the binding between GALNT6 protein and MUC1 protein, and the glycosylation of MUC1 protein by GALNT6 protein as an index of cancer, particularly breast cancer.

### PRIORITY

The present application claims the benefit of U.S. Provisional Application No. 61/275,197, filed on Aug. 25, 2009, the entire contents of which are incorporated by reference herein.

### TECHNICAL FIELD

The present invention relates to methods of screening for a substance suited to the treatment and/or prevention of cancer. In particular, the present invention relates to methods that use or target the interaction between GALNT6 and MUC1 as an index of cancer.

### BACKGROUND ART

Breast cancer is the most common cancer among women worldwide, and more than a million women are diagnosed with breast cancer every year (NPL1). Early detection with mammography as well as the development of molecular-targeted drugs, such as tamoxifen, aromatase inhibitors, and trastuzumab (Herceptin), have contributed a reduction in mortality rate and provided a better quality of life to the patients, particularly those with breast tumors expressing the estrogen receptor (ER) or human epidermal growth factor receptor-2 (HER2). However, a significant portion of patients has no clinical benefit from these treatments. Furthermore, long-term tamoxifen administration has been shown to increase the risk of endometrial cancer and trastuzumab treatment has been linked to cardiac toxicity (NPL2). Hence, development of novel molecular-targeted drugs for breast cancer with higher efficacy and low risk of adverse reactions is essentially important to improve clinical managements.

To that end, the genome-wide gene expression profile of 81 breast cancers as well as 29 normal human organs were analyzed using cDNA microarray and several novel target candidates for breast cancer therapy were reported (NPL3-9). One in particular was UDP-N-acetyl-alpha-D-galactosamine: polypeptide N-acetylgalactosaminyltransferases-6 (GALNT6), which was upregulated in a great majority of breast cancer cases (PTL1).

The mucin-type O-glycosylation is initiated by GALNT family members that transfer N-acetyl-alpha-D-galactosamine (GalNAc) to serine or threonine residues on target proteins (NPL10). This modification occurs in the Golgi complex and is presumably controlled by the expression and distribution of GALNT proteins (NPT11). Interestingly, the structure of glycan chains covalently attached to glycoproteins was altered in breast cancer cells. For instance, the O-glycans were often truncated (core 1-based type) in breast carcinoma cells, whereas the chain was extended (core 2-based type) in normal breast cells (NPT12). Mucin-1 (MUC1), a type I transmembrane protein, is known to contribute to mammary carcinogenesis through interaction with EGFRs (Epidermal Growth Factor Receptors), ER-alpha (Estrogen Receptor alpha), and beta-catenin (NPT13). These aberrant O-type glycosylations have been suggested to regulate the protein stability and subcellular distribution of MUC1(NPT14). However, the mechanism of such aberrant O-glycosylation of proteins in breast cancer cells has been largely unknown.

### CITATION LIST

### Patent Literature

[PTL 1] WO2007/013670

### Non Patent Literature

[NPL1] Parkin, D. M., Bray, F., Ferlay, J. & Pisani, P. Global cancer statistics, 2002. CA Cancer J. Clin. 55, 74-108 (2005).
[NPL2] Moulder, S. & Hortobagyi, G. N. Advances in the treatment of breast cancer. Clin. Pharmacol. Ther. 83, 26-36 (2008).
[NPL3] Nishidate, T. et al. Genome-wide gene-expression profiles of breast-cancer cells purified with laser microbeam microdissection: identification of genes associated with progression and metastasis. Int. J. Oncol. 25, 797-819 (2004).
[NPL4] Saito-Hisaminato, A. et al. Genome-wide profiling of gene expression in 29 normal human tissues with a cDNA microarray. DNA Res. 9, 35-45 (2002).
[NPL5] Park, J. H., Lin, M. L., Nishidate, T., Nakamura, Y. & Katagiri, T. PDZ-binding kinase/T-LAK cell-originated protein kinase, a putative cancer/testis antigen with an oncogenic activity in breast cancer. Cancer Res. 66, 9186-9195 (2006).
[NPL6] Lin, M. L., Park, J. H., Nishidate, T., Nakamura, Y. & Katagiri, T. Involvement of maternal embryonic leucine zipper kinase (MELK) in mammary carcinogenesis through interaction with Bcl-G, a pro-apoptotic member of the Bcl-2 family. Breast Cancer Res. 9, R17 (2007).
[NPL7] Shimo, A. et al. Elevated expression of protein regulator of cytokinesis 1, involved in the growth of breast cancer cells. Cancer Sci. 98, 174-181 (2007).
[NPL8] Shimo, A. et al. Involvement of KIF2C/MCAK overexpression in mammary carcinogenesis. Cancer Sci. 99, 62-70 (2008).
[NPL9] Ueki, T. et al. Involvement of elevated expression of multiple cell-cycle regulator, DTL/RAMP (denticleless/RA-regulated nuclear matrix associated protein), in the growth of breast cancer cells. Oncogene 27, 5672-5683 (2008).
[NPL10] Hagen, K. G. T., Fritz, T. A. & Tabak, L. A. All in the family: the UDP-GalNAc:polypeptide N-acetylgalactosaminyltransferases. Glycobiology 13, 1R-16R (2003)
[NPL11] Brooks, S. A., Carter, T. M., Bennett, E. P., Clausen, H. & Mandel, U. Immunolocalisation of members of the polypeptide N-acetylgalactosaminyl transferase (ppGalNAc-T) family is consistent with biologically relevant altered cell surface glycosylation in breast cancer. Acta Histochem. 109, 273-284 (2007).
[NPL12] Burchell, J. M., Mungul, A. & Taylor-Papadimitriou, J. O-linked glycosylation in the mammary gland: changes that occur during malignancy. J. Mammary Gland Biol. Neoplasia 6, 355-364 (2001).
[NPL13] Carraway, K. L. 3rd, Funes, M., Workman, H. C. & Sweeney, C. Contribution of membrane mucins to tumor progression through modulation of cellular growth signaling pathways. Curr. Top. Dev. Biol. 78, 1-22 (2007).
[NPL14] Altschuler, Y. et al. Clathrin-mediated endocytosis of MUC1 is modulated by its glycosylation state. Mol. Biol. Cell 11, 819-831 (2000).

### SUMMARY OF INVENTION

### Technical Problem

Development of novel molecular-targeted drugs for breast cancer with higher efficacy and low risk of adverse reactions is desired to improve clinical managements.

### Solution to Problem

The structure of glycan chains covalently attached to glycoproteins has been reported to be altered in breast cancer cells. However, the mechanism of such aberrant O-glycosylation of proteins in breast cancer cells has been largely unknown.

The present invention is based on the discovery of a novel drug target, GALNT6, which is upregulated in a great majority of breast cancers and encodes a glycosyltransferase responsible for initiating mucin-type O-glycosylation in mammary carcinogenesis. More particularly, the present invention relates to the interaction between the cancer-related gene GALNT6 and MUC1, both of which are commonly upregulated in tumors, and strategies for the development of molecular targeted drugs for cancer treatment using GALNT6 and MUC1.

As demonstrated herein, Western-blot and immunocytochemical analyses indicated that wild-type GALNT6 protein could glycosylate and stabilize the MUC1 oncoprotein. Immunohistochemical staining analysis further confirmed the coupregulation of GALNT6 and MUC1 proteins in breast cancer specimens. Finally, knockdown of GALNT6 or MUC1 led to similar morphologic changes (round shape and enlarged size) of cancer cells accompanied by the increase of cell adhesion molecules, beta-catenin and E-cadherin.

Accordingly, it is one object of the present invention to provide a method for screening for a substance suitable for use the treatment and/or prevention of cancers expressing GALNT6, such as breast cancer, using as an index or targeting the interaction between the GALNT6 polypeptide and MUC1 polypeptide. It is a further object of the present invention to provide a method of screening for a substance suitable for use the treatment and/or prevention of a cancer expressing GALNT6, such as breast cancer, using as an index or targeting the binding between the GALNT6 polypeptide and MUC1 polypeptide.

It is yet another object of the present invention to provide a method of screening for a substance suitable for use the treatment and/or prevention of a cancer expressing GALNT6, for example, breast cancer, wherein the method includes the steps of: contacting a test substance with a GALNT6 polypeptide, or cell expressing the GALNT6 polypeptide, and selecting the test substance that suppresses the glycosylation level of a MUC1 polypeptide.

It is yet another object of the present invention to provide a method of screening for a substance suitable for use the treatment and/or prevention of a cancer expressing GALNT6, such as breast cancer, wherein the method includes the steps of: contacting a GALNT 6 polypeptide and a MUC polypeptide in a test substance, and selecting the test substance that suppresses the glyxosylation level of the MUC1 polypeptide.

In some embodiments, the GALNT6 polypeptide to be used in the above screening methods includes a histidine 271(H271) and/or glutamic acid 382(E382) residue of SEQ ID NO: 29. In another embodiment, the MUC1 polypeptide to be used in the above screening methods may include a peptide fragment derived from the variable number tandem repeat (VNTR) domain of MUC1 protein including one or more serine residues and/or threonine residues, such as MUC1-a (AHGVTSAPDTR) or MUC1-b(RPAPGSTAPPA).

### Advantageous Effects of Invention

The present invention provides new methods of identifying substances suitable for use in the treatment and/or prevention of cancer. Substances identified by the methods of the present invention may serve as valuable targets in the development of therapeutic modalities against breast cancer.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF DRAWINGS

Various aspects and applications of the present invention will become apparent to the skilled artisan upon consideration of the brief description of the figures and the detailed description of the present invention and its preferred embodiments that follows.

FIG. 1 demonstrates the upregulation of GALNT6 in breast cancer. Parts a-f depict the results of immunohistochemical staining of tissue sections of breast cancer (a), normal breast (b), lung (c), heart (d), liver (e), and kidney (f). Representative figures were from microscopic observations with original magnification, left; ×100 and right; ×200. Part g depicts the subcellular localization of the endogenous GALNT6 protein in T47D cells. Endogenous GALNT6 protein is localized with Golgi-58k, a marker for Golgi complex. DAPI was co-stained to discriminate from the nucleus. Part h depicts the subcellular localization of the endogenous GALNT6 protein in a breast cancer tissue sections. Arrows indicate the Golgi apparatus. Part i depicts the results of semiquantitative RT-PCR of GALNT6 in microdissected tumor cells from 12 breast cancer specimens (upper panels), and in 19 breast cancer cell lines, normal epithelial cell-line (HBL-100) and normal human organs (ductal cells; normal breast ductal cells, M.G.; mammary gland) (lower panels). Expression of GAPDH served as a quantity control. Part j depicts the results of Northern-blot analysis of breast cancer cell lines. The radioisotope-labeled probe of GALNT6 cDNA detected an approximately 5-kb transcript, indicating up-regulated expression of GALNT6 in breast cancer cell-lines.

FIG. 2 depicts the knockdown of GALNT6 in T47D breast cancer cells. In part a, the knockdown effect of GALNT6 expression by shRNAs was confirmed at 10 days after transfection by semiquantitative RT-PCR analysis (upper panels). GAPDH, is a quantity control. MTT assays were graphed after standardization by Mock to 1.0. Asterisk indicates P<0.05 (middle panels). Colony formation assays were carried out after three-week selective incubation (bottom panels). Part b-d depict the knockdown of GALNT6 by siRNA. Part b depicts the knockdown of GALNT6 and corresponding cell morphology four days after the transfection, monitored by western blot (left panels) and microscopic observation (right panels), respectively. In part c-d, each of the cell shapes was further investigated by immunostaining with fluorescence-labeled phalloidin at 4 days after transfection with si-EGFP (c) and si-GALNT6 (d). DAPI was co-stained to discriminate from the nucleus.

FIG. 3 confirms the specificity of anti-GALNT6 antibodies. In parts a-c, Western blot analysis and immunocytochemical staining were carried out using anti-GALNT6 polyclonal antibody (a) or anti-GALNT6 monoclonal antibodies of clone #4H11 (b) and #3G7 (c). Arrows indicate Golgi apparatus.

FIG. 4 confirms that GALNT6 is critical for MUC1 stabilization. In part a, T47D cells were transfected with si-EGFP or si-GALNT6, and collected at days 1, 2 and 4, followed by Western blot (upper panels) and semiquantitative RT-PCR (lower panels). Part b depicts the results of T47D cell co-staining with anti-GALNT6 polyclonal antibody and anti-MUC1 monoclonal antibody four days after the transfection with si-GALNT6 (lower panels) or si-EGFP (upper panels). Part c depicts the results of MCF10A cell co-staining with anti-HA Rat and anti-MUC1 monoclonal antibodies two days after transfection with a GALNT6-construct (pCAGGS-GALNT6-HA). Arrows indicate GALNT6-expressing MCF10A cells. Part d depicts the results of Western blot analysis confirming the co-overexpression of GALNT6 and MUC1 in breast cancer cell lines. Asterisk indicates a human normal breast epithelial cell-line, HMEC.

FIG. 5 depicts the knockdown of GALNT6 and MUC1 in breast cancer cell lines. Part a depicts Western blot results of T47D (left panels), MCF7 (middle panels), SKBR3 (right panels) cells at four days after transfection with each of si-GALNT6, si-MUC1 and si-EGFP (control). Expression of beta-actins served as quantity controls at protein levels. Parts b and c present the results of microscopic observation and a cell proliferation assay of breast cancer cells (T47D, MCF7, and SKBR3) after knockdown of GALNT6 and MUC1. Four days after the transfection with each of si-GALNT6, si-MUC1 and si-EGFP (control), cell morphology and viability were investigated by a phase-contrast microscopy (b) and MTT assay (c), respectively. MTT assays were performed to evaluate cell viability and graphed after standardization by Mock to 1.0. The asterisk indicates the statistical significance with p-value of <0.001 (*) and p<0.0001(**) in the unpaired t-test.

FIG. 6 confirms that GALNT6 O-glycosylates MUC1 protein. Part a depicts the in vitro O-glycosylation of MUC-1a (upper) and MUC1-b (lower) peptides by WT-GALNT6 recombinant protein. Part b depicts the in vitro O-glycosylation of MUC-1a (left) and MUC1-b (right) peptides by WT-GALNT6, H271D or E382Q recombinant proteins after 16 hours reaction. Part c depicts the enzymatic products shown on b were digested by alpha-N-acetylgalactosaminidase (Acremonium sp.) at 37 degrees C. for 20 hours. The digested samples were separated by reversed phase HPLC. Asterisk indicates contaminated materials during HPLC (a-c). Part d depicts the results of Western blot analysis of stably GALNT6 expressing-HeLa cells. Parts e and f confirm the O-glycosylation of MUC1. Representative clones of mock (#003), WT (#110), and H271D (#114) were used for immunoprecipitation with anti-MUC1 monoclonal antibody (e) or pull-down assay using biotin-conjugated VVA lectin and Streptavidinagarose (f). Subsequently, the precipitates were immunoblotted with anti-MUC1 monoclonal antibody and VVA-lectin.

FIG. 6 confirms that GALNT6 O-glycosylates MUC1 protein. Part g depicts the exogenous expression of GALNT6 in MCF10A cells. Two days after the transfection with WT-GALNT6 and H271D plasmids, the cells were co-stained with anti-HA rat and anti-MUC1 monoclonal antibodies.

FIG. 7 depicts the stabilization of the MUC1 protein by GALNT6 in breast cancer cells. Part a depicts the results of Western blot and semi-quantitative RT-PCR analyses of T47D (left panels) and MCF7 (right panels) cells at four days after transfection with each of si-GALNT6, si-MUC1 and si-EGFP (control). Expression of ACTB and beta-actins served as quantity controls at transcriptional and protein levels, respectively. Part b depicts the results of immunocytochemical staining of breast cancer cells (T47D and MCF7) knocked-down with si-GALNT6. Arrows indicate GALNT6-expressing and -depleted cells.

FIG. 8 confirms that GALNT6 and MUC1 are involved in cytoskeletal regulation. Part a depicts the results of Western blot (1^{st} to 5^{th} panels) and semiquantitative RT-PCR (6^{th} to 10^{th} panels) analyses for beta-catenin (CTNNB1) and E-cadherin (CDH1) in si-GALNT6 or si-MUC1-transfected cells. Beta-actin and ACTB, quantity controls at protein and transcriptional levels, respectively. Parts b and c depict the results of immunocytochemical staining of beta-catenin and E-cadherin in si-EGFP or si-GALNT6-transfected T47D cells, using anti-GALNT6 polyclonal antibody, monoclonal antibodies against beta-catenin (b) or E-cadherin (c). Part d depicts a schematic representation of GALNT6-MUC1 pathway in mammary carcinogenesis. Overexpression of GALNT6 attributes to aberrant glycosylation and stabilization of MUC1, and induces the elevated interaction with several signal transducers, and thereby results in proliferation and anti-cell adhesion of breast cancer cells.

FIG. 9 depicts the results of immunohistochemical staining using anti-GALNT6 and -MUC1 monoclonal antibodies. Representative figures of paired normal and cancer tissue sections of the breast (#185, #186 and #187) after immunostaining with anti-GALNT6 (#3G7) and anti-MUC1 (#VU4H5) monoclonal antibodies are presented (microscopic observation; ×100). Both the GALNT6 and MUC1 proteins were specifically expressed in breast cancers cells, but not expressed in normal duct cells.

FIG. 10 demonstrates that the knockdown of MUC1 elevates cell-adhesion complex. Four days after the transfection with si-EGFP (left panels) or si-MUC1 (right panels), T47D cells were immunostained with mouse monoclonal antibodies of MUC1, beta-catenin, and E-cadherin, individually.

FIG. 11 confirms that MUC1 inhibits cell to dish attachment. Four days after transfection with siRNAs (si-EGFP, -GALNT6, and -MUC1) into T47D cells, the strength of cell-to-dish attachment was quantified by the cell detachment assay. Part a depicts cell morphology monitored by a phase-contrast microscopy after transfection with each siRNA. Part b depicts Western blot analysis to assess knockdown of GALNT6 and MUC1 proteins. Part c depicts cell numbers on the plate dish was relatively counted by MTT assays in triplicate. Between the 1st and 2nd MTT assays, the cells were incubated with 5 mM of EDTA in PBS (−) for 10 min to remove any detached cells, and further incubated for 12 hours in fresh culture medium. Part d depicts the percentage of attached cells was counted and graphed (*, p<0.05; **, p<0.01 in the unpaired t-test).

### DESCRIPTION OF EMBODIMENTS

Although any methods and materials similar or equivalent to those described herein can be used in the practice or testing of embodiments of the present invention, the preferred methods, devices and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue of prior invention. In case of conflict, the present specification, including definitions, will control. In addition, the materials, methods, and examples are illustrative only and not intended to be limiting.

### I. DEFINITION

The words “a”, “an”, and “the” as used herein mean “at least one” unless otherwise specifically indicated.

The terms “isolated” and “purified” when used herein in relation to a substance (e.g., polypeptide, antibody, polynucleotide, etc.) indicate that the substance is substantially free from at least one substance that may else be included in the natural source. Thus, an isolated or purified antibody refers to antibodies that is substantially free of cellular material such as carbohydrate, lipid, or other contaminating proteins from the cell or tissue source from which the protein (antibody) is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, antibodies of the present invention are isolated or purified.

An “isolated” or “purified” nucleic acid molecule, such as a cDNA molecule, can be substantially free of other cellular material, or culture medium when produced by recombinant techniques, or substantially free of chemical precursors or other chemicals when chemically synthesized. In a preferred embodiment, nucleic acid molecules encoding antibodies of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to substances that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical substances that have different structures but similar functions to general amino acids. Amino acids may be referred to herein by their commonly known three letter symbols or the one-letter symbols recommended by the IUPAC-IUB Biochemical Nomenclature Commission.

The terms “gene”, “polynucleotide”, “oligonucleotide”, “nucleic acid”, and “nucleic acid molecule” are used interchangeably unless otherwise specifically indicated and, similarly to the amino acids, are referred to by their commonly accepted single-letter codes. Similar to the amino acids, they encompass both naturally-occurring and non-naturally occurring nucleic acid polymers. The polynucleotide, oligonucleotide, nucleic acids, or nucleic acid molecules may be composed of DNA, RNA or a combination thereof.

Unless otherwise defined, the term “cancer” refers to cancer over-expressing the GALNT6 gene. Examples of cancers over-expressing GALNT6 include, but are not limited to, breast cancer.

In the context of the present invention, a substance that suppresses or inhibits the binding and/or glycosylation activity between GALNT6 and MUC1 may find utility in the treatment and/or prevention of cancer. A level of binding or glycosylation is deemed to be “suppressed” or “inhibited” if the relevant level is reduced from the control level (e.g., the level detected in the absence of a test substance) by, for example, 10%, 25%, or 50%; or decreases by more than 1.1 fold, more than 1.5 fold, more than 2.0 fold, more than 5.0 fold, more than 10.0 fold, or more.

The present invention finds utility in connection with the treatment and/or prevention of cancer. In the context of the present invention, a treatment is deemed “efficacious” if it leads to clinical benefit such as, reduction in expression of the GALNT6 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

To the extent that the methods of the present invention find utility in the context of “prevention” and “prophylaxis”, such terms are interchangeably used herein to refer to any activity that reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis can include a wide range of prophylactic therapies aimed at alleviating the severity of the particular disorder, e.g. reducing the proliferation and metastasis of tumors.

The treatment and/or prophylaxis of cancer and/or the prevention of postoperative recurrence thereof include any of the following steps, such as the surgical removal of cancer cells, the inhibition of the growth of cancerous cells, the involution or regression of a tumor, the induction of remission and suppression of occurrence of cancer, the tumor regression, and the reduction or inhibition of metastasis. Effectively treating and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis include 10%, 20%, 30% or more reduction, or stable disease.

### II. GALNT6 and MUC1—GENES AND PROTEINS

Exemplified nucleic acid and polypeptide sequences of the genes of interest in the present invention are shown in the following numbers;

GALNT6: SEQ ID NO: 28 and 29;

MUC1: SEQ ID NO: 30 and 31.

However, one of skill will recognize that gene and protein sequences need not be limited to these sequences and that variants (e.g., functional equivalents and allelic variants) can be used in the present invention as described below. Additional sequence data is available via following GenBank accession numbers;

GALNT6: NM_{—}007210; and

MUC1: NM_{—}002456, NM_{—}001018016, NM_{—}001018017, NM_{—}001044390, NM_{—}001044391, NM_{—}001044392 and also NM_{—}001044393.

The above-mentioned amino acid sequence of GALNT6 polypeptide includes a signal peptide sequence (e.g., 1-34 of SEQ ID NO: 29) and a mature GALNT6 polypeptide does not have a signal peptide. Accordingly, in some embodiment, “GALNT6 polypeptide” refers to a mature GALNT6 polypeptide (e.g., 35-622 of SEQ ID NO: 29) without a signal peptide. GALNT6 polypeptide includes a pp-GalNAc-transferase motif (e.g., 180-485 of SEQ ID NO: 29) responsible for glycosylation of substrates. In preferable embodiments, functional equivalents of GALNT6 polypeptide described bellow include a pp-GalNAc-transferase motif of GALNT6 polypeptide.

MUC1 mature polypeptide includes a 20 amino acids variable number tandem repeat (VNTR) domain, with the number of repeats varying from 20 to 120 in different individuals. Example of an amino acid sequence of MUC1 polypeptide having VNTR domain is shown in SEQ ID NO: 32. For example, when the MUC1 polypeptide has an amino acid sequence of SEQ ID NO: 32, the VNTR domain is located in a region of approximately 126 to 965 of SEQ ID NO:32. Generally, serine residues and/or threonine residues within the VNTR domain are glycosylated in MUC1 polypeptide. Accordingly, peptide fragments derived from the VNTR domain of MUC1 polypeptide that include one or more serine residues and/or threonine residues are preferably used as functional equivalents of MUC1 polypeptide described bellow. Any peptide fragments derived from the VNTR domain of MUC1 polypeptide can be used as functional equivalent of the MUC1 polypeptide so long as they include at least one serine residue or threonine residue capable of being glycosylated. Preferably, such peptide fragments may have 10 or more amino acids.

According to an aspect of the present invention, functional equivalents of GALNT6 and MUC1 are also considered to be “polypeptides” of the present invention. Herein, a “functional equivalent” of a protein is a polypeptide that has a biological activity equivalent to the protein. Namely, any polypeptide that retains the biological ability of the original peptide may be used as such a functional equivalent in the present invention. Such functional equivalents include those wherein one or more amino acids are substituted, deleted, added, and/or inserted to the natural occurring amino acid sequence of the protein. Alternatively, the polypeptide may be composed an amino acid sequence having at least about 80% homology (also referred to as sequence identity) to the sequence of the GALNT6 or MUC1 protein (e.g., SEQ ID NO: 29, 31 or 32), more preferably at least about 90% to 95% homology, even more preferably 96%, 97%, 98% or 99% homology. In other embodiments, the polypeptide can be encoded by a polynucleotide that hybridizes under stringent conditions to the naturally occurring nucleotide sequence of the gene.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to that of the human protein of the present invention, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will vary in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength and pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, the particular condition of hybridization selected for isolating a DNA encoding a polypeptide functionally equivalent to the above human protein can be routinely determined by a person skilled in the art. For example, hybridization may be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42 degrees C., 2×SSC, 0.1% SDS, preferably 50 degrees C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

In general, modification of one, two or more amino acid in a protein will not influence the function of the protein. In fact, mutated or modified proteins (i.e., peptides composed of an amino acid sequence in which one, two, or several amino acid residues have been modified through substitution, deletion, insertion and/or addition) have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Thus, in one embodiment, the peptides of the present invention may have an amino acid sequence of SEQ ID NO: 29, 31 or 32 wherein one, two or even more amino acids are added, inserted, deleted, and/or substituted.

Those of skill in the art will recognize that individual additions, deletions, insertions, or substitutions to an amino acid sequence that alter a single amino acid or a small percentage of amino acids or those considered to be a “conservative modifications”, i.e., one wherein the alteration results in the conservation of properties of the original amino acid side chain(s), tend to result in the generation of a protein having functions similar to those of the original reference protein. As such, they are acceptable in the context of the instant invention.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 5 or 6 amino acids or less, and even more preferably 3 or 4 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins 1984).

Such conservatively modified polypeptides are included in the present protein. However, the present invention is not restricted thereto and includes non-conservative modifications, so long as at least one biological activity of the protein is retained. Furthermore, the modified proteins do not exclude polymorphic variants, interspecies homologues, and those encoded by alleles of these proteins.

Moreover, the gene of the present invention encompasses polynucleotides that encode such functional equivalents of the protein. In addition to hybridization, a gene amplification method, for example, the polymerase chain reaction (PCR) method, can be utilized to isolate a polynucleotide encoding a polypeptide functionally equivalent to the protein, using a primer synthesized based on the sequence above information. Polynucleotides and polypeptides that are functionally equivalent to the human gene and protein, respectively, normally have a high homology to the originating nucleotide or amino acid sequence of. “High homology” typically refers to a homology of 40% or higher, preferably 60% or higher, more preferably 80% or higher, even more preferably 90% to 95% or higher, even more preferably 96% to 99% or higher. The homology of a particular polynucleotide or polypeptide can be determined by following the algorithm in “Wilbur and Lipmann, Proc Natl Acad Sci USA 80: 726-30 (1983)”.

Polypeptides to be used for the screening method of the present invention may be a recombinant polypeptide or a protein derived from the nature or a partial peptide thereof. For example, a purified polypeptide, a soluble protein, a form bound to a carrier or a fusion protein fused with other polypeptides may be used. The methods for preparation of polypeptides are well-known in the art. For example, the gene encoding the polypeptide is expressed in host (e.g., animal) cells and so on by inserting the gene to an expression vector for foreign genes, such as pSV2neo, pcDNA I, pcDNA3.1, pCAGGS and pCD8. The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the polypeptide by the fusion is also reported. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for isolation of the polypeptide (Experimental Medicine 13: 85-90 (1995)).

Alternatively, polypeptides to be used in the present invention may be obtained through chemical synthesis based on the selected amino acid sequence. Examples of conventional peptide synthesis methods that may be adapted for the synthesis include:

(i) Peptide Synthesis, Interscience, New York, 1966;

(ii) The Proteins, Vol. 2, Academic Press, New York, 1976;

(iii) Peptide Synthesis (in Japanese), Maruzen Co., 1975;

(iv) Basics and Experiment of Peptide Synthesis (in Japanese), Maruzen Co., 1985;

(v) Development of Pharmaceuticals (second volume) (in Japanese), Vol. 14 (peptide synthesis), Hirokawa, 1991;

(vi) WO99/67288; and

(vii) Barany G. & Merrifield R. B., Peptides Vol. 2, “Solid Phase Peptide Synthesis”, Academic Press, New York, 1980, 100-118.

Polypeptides may be purified or isolated from cell lysate or reaction mixture used for the production of the polypeptides. Purification or isolation can be conducted according to the conventional methods in the art. For example, column chromatography, filter, ultrafiltration, salt precipitation, solvent precipitation, solvent extraction, distillation, immunoprecipitation, SDS-polyacrylamide gel electrophoresis, isoelectric point electrophoresis, dialysis, and recrystallization may be appropriately selected and combined to isolate and purify the polypeptides. Examples of chromatography include, for example, affinity chromatography, ion-exchange chromatography, hydrophobic chromatography, gel filtration, reverse phase chromatography, adsorption chromatography, and such (Strategies for Protein Purification and Characterization: A Laboratory Course Manual. Ed. Daniel R. Marshak et al., Cold Spring Harbor Laboratory Press (1996)). These chromatographies may be performed by liquid chromatography, such as HPLC and FPLC. Thus, the present invention provides highly purified polypeptides prepared by the above methods.

### III. ANTIBODY

Antibodies are useful in detecting binding between proteins or glycosylated proteins. Accordingly, in some embodiments, antibodies against GALNT6 protein or MUC1 protein or immunogenic fragments of such antibodies may be preferably used in the screening methods of the present invention.

Antibodies against GALNT6 protein or MUC1 protein can be prepared from a GALNT6 protein or MUC1 protein, or an immunogenic fragments thereof ((e.g., a GALNT6 protein corresponding to codons 35-622) (see the Item of ‘Generation of anti-GALNT6 specific antibody’ in EXAMPLE)). Thus, in a preferred embodiment, antibodies of GALNT6 protein may be antibodies that recognize GALNT6 and that binds an epitope including residues 35-622 of the amino acid sequence of SEQ ID NO: 29. Also, in a preferred embodiment, antibodies of MUC1 protein may be antibodies that recognize glycosylated one or more serine and/or threonine residue in the VNTR domain of MUC1 polypeptide.

The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab', F(ab′)₂, Fab, Fv and rIgG). See also, Pierce Catalog and Handbook, 1994-1995 (Pierce Chemical Co., Rockford, Ill.). See also, e.g. Kuby, J., Immunology, 3rd Ed., W.H. Freeman & Co., New York (1998). Such non-naturally occurring antibodies can be constructed using solid phase peptide synthesis, produced recombinantly or obtained, for example, by screening combinatorial libraries of variable heavy chains and variable light chains as described by Huse et al., Science 246:1275-81 (1989), which is incorporated herein by reference. These and other methods of making, for example, chimeric, humanized, CDR-grafted, single chain, and bifunctional antibodies are well known to those skilled in the art (Winter and Harris, Immunol. Today 14:243-6 (1993); Ward et al., Nature 341:544-6 (1989); Harlow and Lane, Antibodies, 511-52, Cold Spring Harbor Laboratory publications, New York, 1988; Hilyard et al., Protein Engineering: A practical approach (IRL Press 1992); Borrebaeck, Antibody Engineering, 2d ed. (Oxford University Press 1995); each of which is incorporated herein by reference).

In the context of the present invention, the term “antibody” includes both polyclonal and monoclonal antibodies. The term also encompasses genetically engineered forms such as chimeric antibodies (e.g., humanized murine antibodies) and heteroconjugate antibodies (e.g., bispecific antibodies). The term further extends to recombinant single chain Fv fragments (scFv) and includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Holliger et al. (1993) Proc Natl Acad Sci USA. 90:6444, Gruber et al. (1994) J Immunol:5368, Zhu et al. (1997) Protein Sci 6:781, Hu et al. (1997) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region; these regions are often referred to as “domains”. Light and heavy chain variable regions contain four “framework” regions interrupted by three hyper-variable regions, also known as “complementarity-determining regions” or “CDRs”. Framework regions and CDRs have been extensively studied and characterized. The sequences of the framework regions of different light and heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab.

The phrase “single chain Fv”, or “scFv”, refers to an antibody in which the variable domains of the heavy and light chains of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (i.e., the variable region) is linked to a constant region of a different or altered class, effector function and/or species, or an entirely different molecule which confers new properties to the chimeric antibody, e.g., an enzyme, toxin, hormone, growth factor, or drug function, etc.; or (b) the variable region, or a portion thereof, is altered, replaced or exchanged with a variable region having a different or altered antigen specificity.

A “humanized antibody” is an immunoglobulin molecule that contains a minimal sequence derived from non-human immunoglobulin. Humanized antibodies include human immunoglobulins (recipient antibody) in which residues from a complementary determining region (CDR) of the recipient are replaced with residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also include residues that are found neither in the recipient antibody nor in the imported CDR or framework sequences. In general, a humanized antibody will contain substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody will optimally also include at least a portion of an immunoglobulin constant region (Fc), typically that of a human immunoglobulin (Jones et al., Nature 321:522-5 (1986); Riechmann et al., Nature 332:323-7 (1988); and Presta, Curr. Op. Struct. Biol. 2:593-6 (1992)). Humanization can be essentially performed following the method of Winter and co-workers (Jones et al., Nature 321:522-5 (1986); Riechmann et al., Nature 332:323-7 (1988); Verhoeyen et al., Science 239:1534-6 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The terms “epitope” and “antigenic determinant” are used interchangeably to refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope generally includes at least 3, and more typically, at least 5 or 8-10 amino acids in a unique spatial conformation. Methods of determining the spatial conformation of epitopes include, for example, X-ray crystallography and 2-dimensional nuclear magnetic resonance. See, e.g., Epitope Mapping Protocols in Methods in Molecular Biology, Vol. 66, Glenn E. Morris, Ed (1996).

The terms “non-antibody binding protein”, “non-antibody ligand” and “antigen binding protein” are used interchangeably to refer to antibody mimics that use non-immunoglobulin protein scaffolds, including adnectins, avimers, single chain polypeptide binding molecules, and antibody-like binding peptidomimetics, as discussed in more detail below.

Other substances have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules having a binding specificity similar to that of the aggregated, but molecularly separate, light and heavy chain variable region of antibodies. The single-chain binding molecule contains the antigen binding sites of both the heavy and light chain variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc. Natl. Acad. Sci. USA 92(14):6552-6556 (1995)) disclose an alternative to antibodies based on cytochrome b562. Ku et al. (1995) generated a library in which two of the loops of cytochrome b562 were randomized and selected for binding against bovine serum albumin. The individual mutants were found to bind selectively with BSA in a fashion similar to anti-BSA antibodies.

Lipovsek et al. (U.S. Pat. Nos. 6,818,418 and 7,115,396) disclose an antibody mimic featuring a fibronectin or fibronectin-like protein scaffold and at least one variable loop. Known as Adnectins, these fibronectin-based antibody mimics exhibit many of the same characteristics of natural and engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions that would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc. Natl. Acad. Sci. USA 96(5):1898-1903 (1999)) disclose an antibody mimic based on a lipocalin scaffold (Anticalin (registered trademark)). Lipocalins are composed of a beta-barrel with four hypervariable loops at the terminus of the protein. Beste (1999) subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin (registered trademark) finds utility as an alternative to antibodies.

Anticalins (registered trademark) are small, single chain peptides, typically between 160 and 180 residues, that provide several advantages over antibodies, including decreased cost of production, increased stability in storage and decreased immunological reaction.

Hamilton et al. (U.S. Pat. No. 5,770,380) disclose a synthetic antibody mimic that combines the rigid, non-peptide organic scaffold of calixarene with multiple variable peptide loops as binding sites. The peptide loops all project from the same geometric side of the calixarene molecule, with respect to each other. Due to this geometric conformation, all of the loops are available for binding and thereby increase the binding affinity to a ligand. However, in comparison to other antibody mimics, the calixarene-based antibody mimic is not restricted exclusively to peptides, and is therefore less vulnerable to attack by protease enzymes. Nor is the scaffold purely of a peptide, DNA or RNA nature, meaning of the antibody mimic is relatively stable in extreme environmental conditions and has a long life span. Further, since the calixarene-based antibody mimic is relatively small, it is less likely to produce an immunogenic response.

Murali et al. (Cell. Mol. Biol. 49(2):209-216 (2003)) discuss a methodology for reducing antibodies into smaller peptidomimetics termed “antibody like binding peptidomimetics” (ABiP) that can also be useful as an alternative to antibodies.

Silverman et al. (Nat. Biotechnol. (2005), 23: 1556-1561) disclose fusion proteins that are single-chain polypeptides having multiple domains termed “avimers”. Developed from human extracellular receptor domains by in vitro exon shuffling and phage display, the avimers are a class of binding proteins somewhat similar to antibodies in their affinities and specificities for various target molecules. The resulting multidomain proteins can include multiple independent binding domains that can exhibit improved affinity (in some cases sub-nanomolar) and specificity as compared to single-epitope binding proteins. Additional details concerning methods of construction and use of avimers are disclosed, for example, in U.S. Patent App. Pub. Nos. 20040175756, 20050048512, 20050053973, 20050089932 and 20050221384, the relevant contents of which are incorporated by reference herein.

In addition to non-immunoglobulin protein frameworks, antibody properties have also been mimicked in substances composed of RNA molecules and unnatural oligomers (e.g., protease inhibitors, benzodiazepines, purine derivatives and beta-turn mimics), all of which are suitable for use with the present invention.

### IV SCREENING METHODS USING THE BINDING BETWEEN GALNT6 AND MUC1 AS AN INDEX OF CANCER

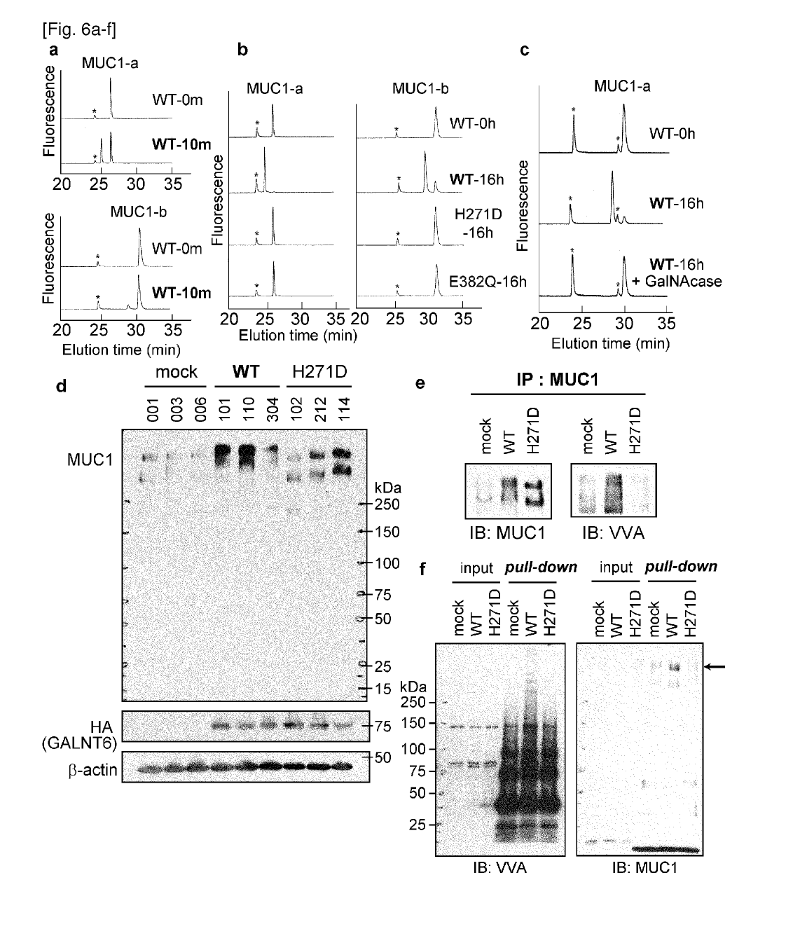

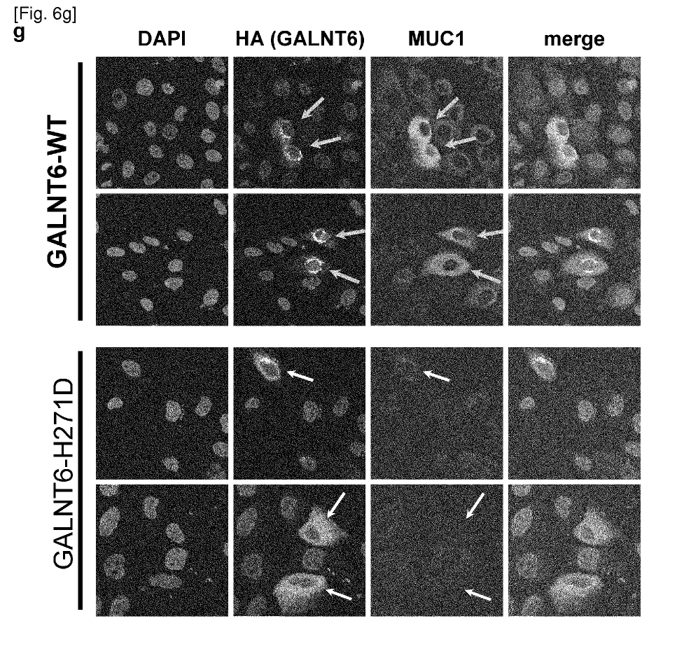

In the present invention, GALNT6 protein was confirmed to interact with MUC1 protein (FIG. 6f). Accordingly, a substance that inhibits the binding between GALNT6 protein and MUC1 protein can be identified using the binding of GALNT6 protein and MUC1 protein as an index. In view thereof, it is an object of the present invention to provide a method of screening for a substance that inhibits the binding between GALNT6 protein and MUC1 protein, such the binding of the GALNT6 protein and MUC1 protein as an index. The present invention also provides a method of screening for a candidate substance that inhibits or reduces the growth or adhesion of breast cancer cells, and a candidate substance for treating or preventing cancers, e.g. breast cancer.

Accordingly, the present invention provides the following methods of [1] to [7]:

[1] A method of screening for a substance that interrupts the binding between a GALNT6 polypeptide and MUC1 polypeptide, such method including the steps of:

(a) contacting a GALNT6 polypeptide or functional equivalent thereof with a MUC1 polypeptide or functional equivalent thereof in the presence of a test substance;

(b) detecting a binding level between the polypeptides;

(c) comparing the binding level detected in the step (b) with those detected in the absence of the test substance; and

(d) selecting the test substance that reduces or inhibits the binding level between the polypeptides;

[2] A method of screening for a candidate substance suitable for the treatment and/or prevention of cancer or that inhibits the binding between a GALNT6 polypeptide and MUC1 polypeptide, such method including the steps of:

(a) contacting a GALNT6 polypeptide or functional equivalent thereof with a MUC1 polypeptide or functional equivalent thereof, in the presence of a test substance;

(b) detecting the binding level between the polypeptides;

(c) comparing the binding level detected in the step (b) with those detected in the absence of the test substance; and

(d) selecting the test substance that inhibits the binding level between the polypeptides;

[3] The method of [1] or [2], wherein the functional equivalent of GALNT6 polypeptide includes an amino acid sequence of a MUC1-binding domain of GALNT6 polypeptide;

[4] The method of [1] or [2], wherein the GALNT6 polypeptide includes the amino acid sequence of amino acid 35 to 622 of SEQ ID NO: 29;

[5] The method of [1] or [2], wherein the functional equivalent of MUC1 polypeptide includes an amino acid sequence of a GALNT6 binding domain of MUC1 polypeptide;

[6] The method of [1] or [2], wherein the functional equivalent of MUC1 includes a peptide derived from a variable number tandem repeat (VNTR) domain of the MUC1 polypeptide; and

[7] The method of claim [2], wherein the cancer is breast cancer.

According to the present invention, the therapeutic effect of a candidate substance on the inhibition of the cell growth or a candidate substance in connection with the treatment and/or prevention of cancer may be evaluated. Therefore, the present invention also provides a method of screening for a candidate substance that suppresses the proliferation of cancer cells, and a method of screening for a candidate substance suited to the treatment and/or prevention cancer.

An illustrative example of such a method includes the steps of:

(a) contacting a GALNT6 polypeptide or functional equivalent thereof with a MUC1 polypeptide or functional equivalent thereof in the presence of a test substance;

(b) detecting the level of binding between the polypeptides;

(c) comparing the binding level detected in the step (b) with those detected in the absence of the test substance; and

(d) correlating the binding level of (c) with the therapeutic effect of the test substance.

Alternatively, in other embodiments, the present invention may provide a method for evaluating or estimating the therapeutic effect of a test substance in connection with the treatment and/or prevention of cancer or the inhibition of cancer, the method including steps of:

(a) contacting a GALNT6 polypeptide or functional equivalent thereof with a MUC1 polypeptide or functional equivalent thereof in the presence of a test substance;

(b) detecting a binding level between the polypeptides;

(c) comparing the binding level detected in the step (b) with those detected in the absence of the test substance; and

(d) correlating the potential therapeutic effect and the test substance, wherein the potential therapeutic effect is shown, when a test substance reduces the binding level.

In the context of the present invention, therapeutic effect may be correlated with the binding level of the GALNT6 and MUC1 proteins. For example, when a test substance reduces the binding level of GALNT6 and MUC1 proteins as compared to a level detected in the absence of the test substance, the test substance may identified or selected as a candidate substance having the desired therapeutic effect. Alternatively, when the test substance does not reduce the binding level of GALNT6 and MUC1 proteins as compared to a level detected in the absence of the test substance, the test substance may identified as the substance having no significant therapeutic effect.

Illustrative candidate substance can include, for example, an inhibitory oligonucleotide (e.g., an antisense oligonucleotide, an siRNA or a ribozyme), an antibody, a polypeptide or a small organic molecule. Screening for suitable inhibitory substances can be carried out using high throughput methods, by simultaneously screening a plurality of substances using multiwell plates (e.g., 96-well, 192-well, 384-well, 768-well, 1536-well). Automated systems for high throughput screening are commercially available from, for example, Caliper Life Sciences, Hopkinton, Mass. Small organic molecule libraries available for screening can be purchased, for example, from Reaction Biology Corp., Malvern, Pa.; TimTec, Newark, Del.

In the context of the present invention, a functional equivalent of a GALNT6 polypeptide will have a biological activity equivalent to a GALNT6 polypeptide (SEQ ID NO:29) (see, Cancer-related genes and cancer-related protein, and functional equivalent thereof in Definition).

In the context of screening for substances that modulate, e.g. inhibit, the binding of GALNT6 polypeptide to MUC1 polypeptide, many methods well known by one skilled in the art can be used.

A polypeptide to be used for screening can be a recombinant polypeptide or a protein derived from natural sources, or a partial peptide thereof. Any test substance aforementioned can be used for screening.

As a method of screening for proteins, for example, that bind to a polypeptide using a GALNT6 and a MUC1 polypeptide or a functional equivalent thereof, many methods well known by a person skilled in the art can be used. Such a screening can be conducted via, for example, an immunoprecipitation, West-Western blotting analysis (Skolnik et al., Cell 65: 83-90 (1991)), a two-hybrid system utilizing cells (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”), affinity chromatography and A biosensor using the surface plasmon resonance phenomenon. Any aforementioned test substance can be used.

In some embodiments, the present screening method may be carried out in a cell-based assay using cells expressing both of a GALNT6 protein and a MUC1 protein. Cells expressing GALNT6 protein and MUC1 protein include, for example, cell lines established from cancer, e.g. breast cancer. Alternatively, the cells may be prepared through transformation with nucleotides encoding GALNT6 and MUC1 protein. Such transformation may be carried out using an expression vector encoding both GALNT6 and MUC1 protein, or expression vectors encoding either GALNT6 or MUC1 protein. The present screening can be conducted by incubating such cells in the presence of a test substance. The binding of GALNT6 protein to MUC1 protein can be detected by immunoprecipitation assay using an anti-GALNT6 antibody or anti-MUC1 antibody (FIG. 6).

In the present invention, it is revealed that suppression of the binding between GALNT6 and MUC1 protein lead to suppression of the growth of cancer cells. Therefore, when a substance inhibits the binding between GALNT6 and MUC1 protein, the inhibition is indicative of a potential therapeutic effect in a subject. In the present invention, a potential therapeutic effect refers to a clinical benefit with a reasonable expectation. In the present invention, such clinical benefit may include;

(a) a reduction of the binding between GALNT6 and MUC1,

(b) a decrease in size, prevalence, or metastatic potential of the cancer in the subject,

(c) the prevention of further cancer formation, or

(d) the prevention or alleviation of a clinical symptom of cancer.

### V. SCREENING METHODS USING THE GLYCOSYLATION LEVEL OF MUC1 BY GALNT6 AS AN INDEX OF CANCER

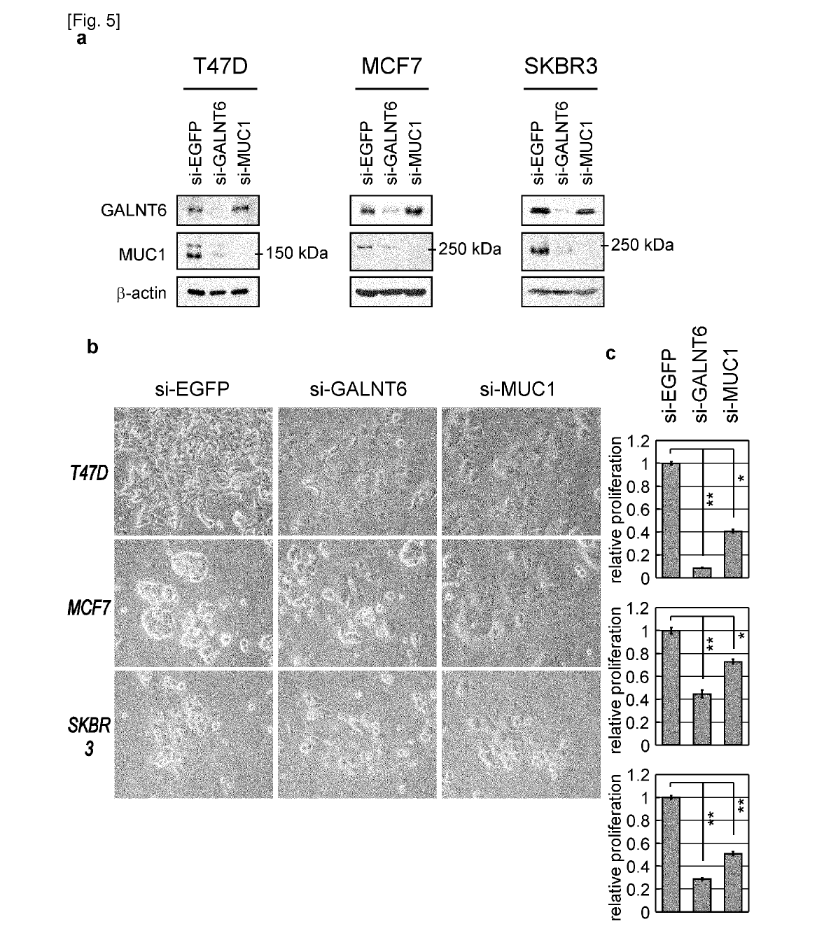

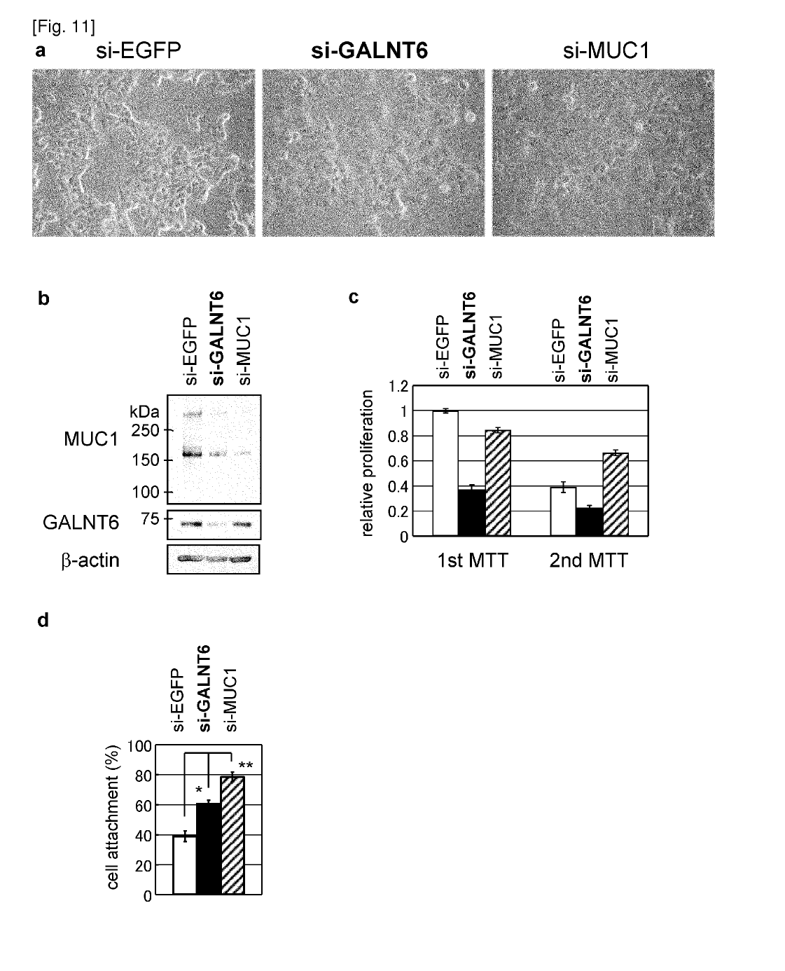

The present invention further confirmed that the MUC1 protein is glycosylated by GALNT6 protein (FIG. 6). The present invention reported critical roles of a novel drug target, GALNT6, that is upregulated in a great majority of breast cancers and encodes a glycosyltransferase responsible for initiating mucin-type O-glycosylation in mammary carcinogenesis. Additionally, knockdown of GALNT6 or MUC1 gene by small-interfering RNA (siRNA) significantly enhanced cell adhesion function (FIG. 11d) and suppressed the growth of breast cancer cells (FIG. 5c). Western-blot and immunocytochemical analyses indicated that wild-type GALNT6 protein could glycosylate and stabilize an oncoprotein MUC1. Immunohistochemical staining analysis confirmed co-upregulation of GALNT6 and MUC1 proteins in breast cancer specimens. Furthermore, knockdown of GALNT6 or MUC1 gene led to similar morphologic changes (round shape and enlarged size) of cancer cells accompanied by the increase of cell adhesion molecules, beta-catenin and E-cadherin. Taken together, these results indicate that overexpression of GALNT6 may contribute to mammary carcinogenesis through aberrant glycosylation and stabilization of MUC1 protein.

Thus, a substance that inhibits the glycosylation of MUC1 protein by GALNT6 protein can be used to inhibit or reduce a growth of cancer cells expressing GALNT6, and can further be useful for inducing apoptosis to cancer cells, or for treating or preventing cancers expressing GALNT6. In the context of the present invention, the preferred target cancer is breast cancer. Therefore, it is a further object of the present invention to provide a method of screening for a substance that inhibits the glycosylation of a MUC1 protein by a GALNT6 protein. Furthermore, the present invention also provides a method of screening for a candidate substance that inhibits or reduces a growth of cancer cells expressing GALNT6, and a candidate substance that induces apoptosis in cancer cells expressing GALNT6. The methods of the present invention are particularly suited to screening for candidate substances having utility in the treatment and/or prevention of cancer, particularly cancers expressing GALNT6. A preferred example of such a cancer is breast cancer.

Accordingly, the present invention provides the following methods of [1] to [12]:

[1]. A method of screening for a candidate substance suitable for the treatment and/or prevention of a cancer or that inhibits the glycosylation of a substrate by the GALNT6 polypeptide, such method including the steps of:

a. incubating GALNT6 polypeptide or functional equivalent thereof and a substrate in the presence of a test substance under a condition suitable for the glycosylation of the substrate by the GALNT6 polypeptide, wherein the functional equivalent is a polypeptide selected from the group consisting of:

i. a polypeptide having the amino acid sequence of SEQ ID NO: 29;

ii. a polypeptide having the amino acid sequence of SEQ ID NO: 29, wherein one or more amino acids are substituted, deleted, or inserted, provided the resulting polypeptide has a biological activity equivalent to the polypeptide of SEQ ID NO: 29; and

iii. a polypeptide encoded by a polynucleotide that hybridizes under stringent conditions to the polynucleotide of SEQ ID NO: 28, provided the resulting polypeptide has a biological activity equivalent to the polypeptide of SEQ ID NO: 29;

b. detecting a substrate glycosylation level; and

c. comparing the substrate glycosylation level to a control level, wherein an increase or decrease in the glycosylation level as compared to said control level indicates that the test substance modulates the glycosylation activity of GALNT6 for the substrate;

[2]. The method of [1], wherein the functional equivalent of the GALNT6 polypeptide is a fragment derived from the polypeptide having the amino acid sequence of SEQ ID NO: 29;

[3]. The method of [2], wherein the fragment includes residues H271 or E382;

[4]. The method of [3], wherein the functional equivalent of the GALNT6 polypeptide includes a pp-GalNAc-transferase motif of the GALNT6 polypeptide;

[5]. The method of [4], wherein the pp-GalNAc-transferase motif includes an amino acid sequence of 180 to 485 of SEQ ID NO:29;

[6]. The method of [1], wherein the GALNT6 polypeptide includes an amino acid sequence of 35 to 622 of SEQ ID NO: 29;

[7]. The method of any one of [1] to [6], wherein the substrate is a MUC1 polypeptide or functional equivalent thereof;

[8]. The method of [7], wherein the MUC1 polypeptide has an amino acid sequence of SEQ ID NO: 31 or 32;

[9]. The method of [7] or [8], wherein the functional equivalent of the MUC1 polypeptide includes a peptide fragment derived from the VNTR domain of the MUC1 polypeptide, wherein the peptide fragment includes one or more serine residues and/or threonine residues;

[10]. The method of [9], wherein the peptide fragment has 10 or more amino acids;

[11]. The method of [7], wherein the functional equivalent is a polypeptide having the amino acid sequence of SEQ ID NO:26(MUC1-a) or SEQ ID NO: 27(MUC1-b); and

[12]. The method of any one of [1] to [11], wherein the glycosylation type is o-glycosylation.

Alternatively, in some embodiments, the present invention may provide a method for evaluating or estimating a therapeutic effect of a test substance in connection with the treatment and/or prevention of cancer or the inhibition of a cancer associated with over-expression of GALNT6, the method including steps of:

(a) contacting a GALNT6 polypeptide or functional equivalent thereof with a substrate to be glycosylated in the presence of the test substance under the condition capable of glycosylation of substrate by GALNT6 polypeptide

(b) detecting the glycosylation level of the substrate; and

(c) correlating the potential therapeutic effect and the test substance, wherein the potential therapeutic effect is shown, when a test substance decreases the glycosylation level of the substrate as compared to the glycosylation level detected in the absence of the test substance as the candidate substance.

In the context of the present invention, the therapeutic effect may be correlated with a glycosylation level of a substrate by GALNT6 polypeptide. For example, when a test substance reduces the glycosylation level of a substrate as compared to a level detected in the absence of the test substance, the test substance may identified or selected as the candidate substance having the therapeutic effect. Alternatively, when the test substance does not reduce the glycosylation level of the substrate as compared to a level detected in the absence of the test substance, the test substance may identified as the substance having no significant therapeutic effect.

It is herein confirmed that GALNT6 protein mediates the glycosylation of MUC1 protein that, in turn, leads to the stabilization of MUC1 protein (see Example 4). After glycosylation, MUC1 protein is accumulated in cancer cells, since MUC1 protein level is enhanced by exogenously expression of GALNT6 (see Example 4). Meanwhile, previous reports suggest that MUC1 captures beta-catenin through interaction with its cytoplasmic tail and thereby inhibits complex formation of cell adhesion molecules (Yuan, Z., Wong, S., Borrelli, A. & Chung, M. A. Biochem. Biophys. Res. Commun. 362, 740-746 (2007), Schroeder, J. A., Adriance, M. C., Thompson, M. C., Camenisch, T. D. & Gendler, S. J. Oncogene 22, 1324-1332 (2003)). Abnormalities of such cell adhesion molecules result in the promotion or mediation of metastasis, invasion, and/or migration of cancer. However, little is known about underlying mechanism for glycosylation of MUC1 protein. The present invention confirms that GALNT6 protein mediates the glycosylation of MUC1 protein. Therefore, a substance that inhibits the glycosylation of a MUC1 protein mediated by GALNT6 protein may be useful for inhibiting or reducing cell growth of cancer. GALNT6 polypeptide and substrate polypeptide (e.g., MUC1 polypeptide) to be used for the screening can be a recombinant polypeptide or a protein derived from natural sources, or a partial peptide thereof. Such polypeptides can be prepared by methods well known in the art (see “II. GALNT6 and MUC1—genes and proteins”). Preferably, the polypeptides is purified or isolated.

In some embodiment, the polypeptides may be added commercially available epitopes to the N- and/or C-terminus. Examples of such epitopes include, but are not limited to, polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such.

In addition to purified or isolated polypeptides, cells that express GALNT6 polypeptide and a substrate polypeptide (e.g., MUC1 polypeptide) may be also used for the screening method of the present invention. Herein, any cell can be used, so long as it expresses the GALNT6 polypeptide or a functional equivalent thereof (see, the “Genes and Proteins” section and definitions above). The cell used in the present screening can be a cell naturally expressing the GALNT6 polypeptide including, for example, cells derived from and cell-lines established from breast cancer. Cell-lines of breast cancer cell, T47D, MCF7, SKBR3 and so on, can be employed.

Alternatively, the cell used in the screening can be a cell that naturally does not express the GALNT6 polypeptide and which is transfected with a GALNT6 polypeptide or a GALNT6 functional equivalent-expressing vector. Such recombinant cells can be obtained through known genetic engineering methods (e.g., Morrison DA., J Bacteriology 1977, 132: 349-51; Clark-Curtiss & Curtiss, Methods in Enzymologist (eds. Wu et al.) 1983, 101: 347-62) as mentioned above.

Any of the aforementioned test substances can be used in connection with the screening methods of the present invention. In some embodiments, substances that can permeate into a cell are selected. Alternatively, when the test substance is a polypeptide, the contact of a cell and the test substance in the present screening can be performed by transforming the cell with a vector that contains the nucleotide sequence coding for the test substance and expressing the test substance in the cell.

In the context of the present invention, as mentioned above, one of the primary biological activities of the GALNT6 protein is glycosylation activity. Glycosylation level of a substrate can be determined by methods known in the art. For example, glycosylation of the substrate may be detected by comparing the molecular weight. Molecular weight of a glycosylated protein is larger than that of predicted size caluculated from the amino acid sequence of the polypeptide by addition of glycoside chain. Furthermore, when the molecular weight of glycosylated protein might be reduced by glycosidase treatment, it was confirmed that the difference of the molecular is caused by addition of glycoside chain. Methods for estimating a molecular weight of a protein are well known.

Alternatively, radiolabeled donor for glycosylation may be used for detection the addition of glycoside chain to the polypeptide. Transfer of the radiolabel to the substrate polypeptide can be detected, for example, by SDS-PAGE electrophoresis and fluorography. Alternatively, following the glycosylation reaction, the substrate can be separated from the glycosyl donor by filtration, and the amount of radiolabel retained on the filter quantitated by scintillation counting. Other suitable labels that can be attached to glycosyl donor, such as chromogenic and fluorescent labels, and methods of detecting transfer of these labels to the substrate, are known in the art. For example, fluorescent labels may preferably used, for example, according to the method described in Examples.

Alternatively, glycosylation level of a substrate can be determined reagents that selectively recognize glycosylated level of the polypeptide. For example, after incubation of the substrate polypeptide and GALNT6 polypeptide, the glycosylation level of the substrate can be detected by immunological method. Any immunological techniques using an antibody recognizing glycosylated polypeptide can be used for the detection. For example, an antibody against glycosylated polypeptide is commercial available. ELISA or Immunoblotting with antibodies recognizing glycosylated polypeptide can be used for the present invention.

Instead of using antibodies, glycosylated protein can be detected using reagents that selectively bind glycoside chain with high affinity. Such reagents are known in the art or can be determined by screening assays known in the art. For example, lectins are well known as glycoside chain specific probe. Lectin reagent conjugated with detectable label such as alkaline-phosphatase is also cmmercialy available.

Glycosylation level of substrate polypeptide in a cell may be estimated by separation of cell lysate. For example, SDS-polyacrylamide gel can be used as the separation of the polypeptide. The polypeptide separated in the gels is transferred to nitrocellulose membranes for immunoblotting analysys.

VI-1. Identifying Therapeutic Substances and Agents:

The level of binding between a GALNT6 polypeptide and MUC1 polypeptide, or the level of glycosylation of a substrate by a GALNT6 polypeptide disclosed herein can also be used to identify candidate therapeutic substances or agents for treating cancer, particularly breast cancer. The methods of the present invention may therefore involve the step of screening a candidate therapeutic substance or agent to determine if the test substance can convert a binding level between a GALNT6 polypeptide and MUC1 polypeptide, or glycosylation level of a substrate by the GALNT6 polypeptide that is characteristic of a cancer state to a binding level between a GALNT6 polypeptide and MUC1 polypeptide, or glycosylation level of a substrate by the GALNT6 polypeptide that is characteristic of a non-cancer state. In the context of such a method, a test cell population or purified polypeptides (i.e., GALNT6 polypeptide and a substrate polypeptide) may be exposed to a test substance or a plurality of test substances (sequentially or in combination) and a binding level between a GALNT6 polypeptide and MUC1 polypeptide, or glycosylation level of a substrate by the GALNT6 polypeptide (in the cells) may be measured. The binding level between a GALNT6 polypeptide and MUC1 polypeptide, or glycosylation level of a substrate by the GALNT6 polypeptide assayed (in the test cell population) is compared to the binding level between a GALNT6 polypeptide and MUC1 polypeptide, or glycosylation level of a substrate by the GALNT6 polypeptide in a normal control (a reference cell population) that is not exposed to the test substance.

A substance that suppresses the binding between a GALNT6 polypeptide and MUC1 polypeptide, or the glycosylation of a substrate by GALNT6 polypeptide has marked clinical benefit. Such substances can be further tested for the ability to forestall or prevent cancer growth in animals or test subjects.

Examples of cells expressing the GALNT6 gene or MUC1 gene include, but are not limited to, cell lines established from breast cancer; such cells can be used for the above screening of the present invention.

Polypeptides for use in the screening methods of the present invention can be obtained as a recombinant protein using the known nucleotide sequence for the GALNT6 gene or the MUC1 gene. In the present invention, other biological activities of the GALNT6 polypeptide or MUC1 polypeptide may be used as indices for further screening to evaluate therapeutic effect of the substances identified by the aforementioned screening method. Based on the information regarding the GALNT6 gene or the MUC1 gene and their encoded proteins, one skilled in the art can select any biological activity of the protein as an index for such screening and any suitable measurement method to assay for the selected biological activity. Specifically, the GALNT6 and MUC1 proteins are known to have a cell proliferating activity, and anti-cell adhesion activity. Therefore, the biological activity can be determined using as an index such cell proliferating activity, and/or anti-cell adhesion activity.

Anti-cell adhesion activity includes any of the following activities, such as;

inhibition of the binding of a cell to a surface, extracellular matrix or another cell, or enhancement of abnormalities of cell adhesion molecules like beta-catenin and E-cadherin.

When the biological activity to be detected in connection with a method of the present invention is cell proliferation or anti-cell adhesion, it can be detected, for example, by preparing cells that express the polypeptide of the present invention, culturing the cells in the presence of a test substance, and determining the count of cell proliferation, or measuring the anti-cell adhesion activity and such, as well as by measuring the colony forming activity, or cell detachment assay as described in the Examples.

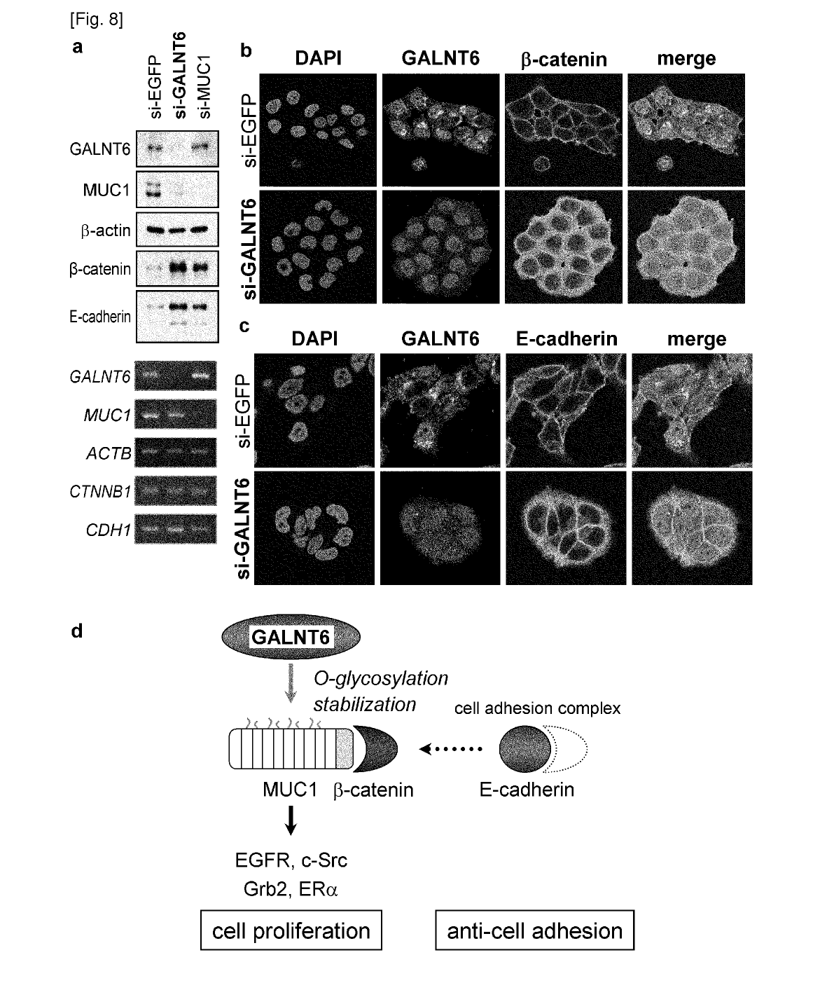

The present invention is the first to reveal that the GALNT6-MUC1 pathway plays a very significant role in stabilization and localization of these two molecules, and formation of the cell adhesion complex (FIG. 8d). In particular, up-regulation of GALNT6 causes stabilization of MUC1 protein through its glycosylation activity. Subsequently, the accumulation of glycosylated MUC1 protein may induce the abnormalities of the cell adhesion molecules like beta-catenin and E-cadherin, resulting in the anti-adhesive effect promoting or mediating metastasis, invasion, and/or migration of cancer. Accordingly, for example, a substance that interferes GALNT6-MUC1 pathway e.g., inhibiting the glycosylation of MUC1 protein has the suppression effect of the anti-adhesive effect. Such substance may thus find utility in the inhibition of metastasis, invasion, and/or migration of cancer. Accordingly, in another embodiment, through the screening methods of the present invention, a candidate substance that inhibits metastasis, invasion, and/or migration of cancer, that interferes GALNT6-MUC1 pathway can also be identified. In other words, the present invention further provides methods for identifying a candidate substance that inhibits or suppresses at least one malignant phenotype selected from the group consisting of metastasis, invasion, and migration of cancer.

VI-2. Selecting a Therapeutic Substance or Agents for Treating Cancer:

Differences in the genetic makeup of individuals can result in differences in their relative abilities to metabolize various drugs. A substance that is metabolized in a subject to act as an anti-cancer substance can manifest itself by inducing a change in a gene expression pattern in the subject's cells from that is characteristic of a cancerous state to a gene expression pattern that is characteristic of a non-cancerous state. Accordingly, the differentially expressed GALNT6 gene, the differential binding between GALNT6 protein and MUC1 protein, and the differential glycosylation level of MUC1 protein by GALNT6 protein allow for a putative therapeutic or prophylactic inhibitor of cancer to be tested in a test cell population from a selected subject in order to determine if the substance is a suitable inhibitor of cancer in the subject, e.g. breast cancer.

To identify an inhibitor of cancer that is appropriate for a specific subject, a test cell population from the subject is exposed to a therapeutic substance or agent, and the binding level between GALNT6 protein and MUC1 protein or the glycosylation level of MUC1 protein by GALNT6 protein is determined.

In the context of the methods of the present invention, the test cell population contains cancer cells expressing the GALNT6 gene. Preferably, the test cell population includes epithelial cells. For example, a test cell population can be incubated in the presence of a candidate substance and the pattern of gene expression of the test cell population can be measured and compared to one or more reference expression profiles, e.g., a cancer reference expression profile, a cancer reference expression profile or normal reference expression profile, e.g., a non-cancer reference expression profile. A decrease in the expression of the GALNT6 gene, the binding level between GALNT6 protein and MUC1 protein and the glycosylation level of MUC1 protein by GALNT6 protein in a test cell population relative to a reference cell population containing cancer indicates that the substance has therapeutic utility. Alternatively, a similarity in the expression of the MUC1 gene, the binding level between GALNT6 protein and MUC1 protein and the glycosylation level of MUC1 protein by GALNT6 protein in the test cell population and the reference cell population indicates that the substance has alternate therapeutic utility.

V-3. Candidate Substances:

In the context of the present invention, the test substance can be any substance or composition. Exemplary test substances include, but are not limited to, immunomodulatory substances (e.g., antibodies), inhibitory oligonucleotides (e.g., antisense oligonucleotides, short-inhibitory oligonucleotides and ribozymes) and small organic substances.

A substance isolated by the screening assays of the present invention may serve as a candidate for the development of anti-cancer drugs and be expected to be applied to the treatment or prevention of breast cancer.

Moreover, substances in which a part of the structure of the substance inhibiting the binding level between GALNT6 protein and MUC1 protein or the glycosylation level of a substrate (e.g., MUC1 protein) by GALNT6 protein is converted by addition, deletion and/or replacement are also included as the substances obtainable by the screening methods of the present invention.

A substance isolated by the screening methods of the present invention has the potential to treat or prevent cancers, or inhibit metastasis, invasion, and/or migration of cancer. Potential of these candidate substances to treat or prevent cancers, or inhibit metastasis, invasion, and/or migration of cancer may be evaluated by second and/or further screening to identify therapeutic substances or agents for cancer

V-4. Screening Kits:

The present invention also provides an article of manufacture or kit containing materials suited to screening for a candidate substance useful in the treatment and/or prevention of cancer, particularly breast cancer. Such an article of manufacture or kit may include one or more labeled containers of materials described herein along with instructions for use. Examples of suitable containers include, but are not limited to, bottles, vials, and test tubes. The containers may be formed from a variety of materials such as glass or plastic.

In one embodiment, the screening kit includes: (a) a first polypeptide including a GALNT6 polypeptide or functional equivalent thereof; (b) a second polypeptide including a MUC1 polypeptide or functional equivalent thereof, and (c) means (e.g., a reagent) to detect the interaction between the first and second polypeptides.

In some embodiments, the functional equivalent of GALNT6 polypeptide includes an amino acid sequence of the MUC1 binding domain of GALNT6 polypeptide. Similarly, in other embodiments, the functional equivalent of MUC1 polypeptide includes an amino acid sequence of the GALNT6 binding domain of MUC1 polypeptide.

In another embodiment, the GALNT6 polypeptide has the amino acid sequence of SEQ ID NO: 29. In another embodiment, the GALNT6 polypeptide has the amino acid sequence of 35 to 622 of SEQ ID NO: 29.

In another embodiment, the screening kit includes: (a) GALNT6 polypeptide or functional equivalent thereof; (b) a substrate, and (c) means (e.g., a reagent) to detect the substrate glycosylation level.

In another embodiment, the GALNT6 polypeptide includes residues H271 or E382.

In another embodiment, the GALNT6 is a fragment having the amino acid sequence of SEQ ID NO: 29. In another embodiment, the GALNT6 polypeptide has the amino acid sequence of 35 to 622 of SEQ ID NO: 29. In a preferred embodiment, the functional equivalent of the GALNT6 polypeptide includes a pp-GalNAc-transferase motif (e.g., 180-485 of SEQ ID NO: 29) of the GALNT6 polypeptide.

In another embodiment, the substrate is a MUC1 polypeptide (e.g., SEQ ID NO: 31 or 32) or functional equivalent thereof. In a preferred embodiment, the functional equivalent of the MUC1 polypeptide includes a peptide fragment derived from the VNTR domain of the MUC1 polypeptide that include one or more serine residues and/or threonine residues. Preferably, such peptide fragment has 10 amino acids or more. For example, peptide having the amino acid sequence of SEQ ID NO: 26(MUC1-a) or SEQ ID NO: 27(MUC1-b) may be preferably used as such peptide fragments. In some embodiments, GALNT6 polypeptide and MUC1 polypeptide are expressed in a living cell.

The present invention further provides articles of manufacture and kits containing materials useful for treating the pathological conditions described herein are provided. Such an article of manufacture may include a container of a medicament as described herein with a label. As noted above, suitable containers include, for example, bottles, vials, and test tubes. The containers may be formed from a variety of materials such as glass or plastic. In the context of the present invention, the container holds a composition having an active substance that is effective for treating a cell proliferative disease, for example, breast cancer. The active substance in the composition may be an identified test substance (e.g., antibody, small molecule, etc.) capable of disrupting the GALNT6/MUC1 association in vivo. The label on the container may indicate that the composition is used for treating one or more conditions characterized by abnormal cell proliferation, or anti-cell adhesion. The label may also indicate directions for administration and monitoring techniques, such as those described herein.

In addition to the container described above, a kit of the present invention may optionally include a second container housing a pharmaceutically-acceptable diluent. It may further include other materials desirable from a commercial end-user standpoint, including other buffers, diluents, filters, needles, syringes, and package inserts with instructions for use.

The compositions may, if desired, be presented in a pack or dispenser device that may contain one or more unit dosage forms containing the active ingredient. The pack may, for example, include a metal or plastic foil, such as a blister pack. The pack or dispenser device may be accompanied by instructions for administration. Compositions including a substance, identified by the screening method of the present invention, formulated in a compatible pharmaceutical carrier may also be prepared, placed in an appropriate container, and labeled for treatment of an indicated condition.

Unless otherwise defined, all technical and scientific terms used herein have the same meaning as commonly understood by one of ordinary skill in the art to which this invention belongs. In case of conflict, the present specification, including definitions, will control.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

### EXAMPLES

### Example 1

### Materials and Methods

Cell-Lines and Clinical Samples:

Human breast cancer cell-lines (BT-20, HCC1937, MCF7, MDA-MB-231, MDA-MB-435S, SKBR3, T47D, YMB-1, BT-474, BT-549, HCC1143, HCC1500, HCC1599, MDA-MB-157, MDA-MB-453, OUCB-F and ZR-75-1), an immortalized human mammary cell-line HBL-100, a monkey kidney cell-line COS-7, a human embryonic kidney fibroblast cell-line HEK293T, and a human cervical carcinoma cell-line, HeLa were purchased from American Type Culture Collection (ATCC, Rockville, Md.) and cultured under their respective depositors' recommendation. HBC-4 and HBC-5 cell-lines were kindly provided by Dr. Takao Yamori of Division of Molecular Pharmacology, Cancer Chemotherapy Center, Japanese Foundation for Cancer Research. Human normal breast epithelial cell-lines (HMEC and MCF10A) were purchased from Cambrex Bioscience Inc (Walkersville, Md.). Tissue samples from surgically-resected breast cancers, and their corresponding clinical information were obtained from the First Department of Surgery, Sapporo Medical University, Hokkaido, and Department of Breast Surgery, The Cancer Institute Hospital of Japanese Foundation for Cancer Research, Tokyo after obtaining written informed consents. This study, as well as the use of all clinical materials described above, was approved by individual institutional Ethical Committees.

Semi-Quantitative Reverse-Transcription Polymerase-Chain Reaction (RT-PCR) and Northern Blot Analyses:

The cDNA from the extracted total RNA of breast cancer cell-lines and clinical samples were prepared as previously described (Park, J. H. et al., Cancer Res. 66, 9186-9195 (2006)). The PCR primer sequences were 5′-CGACCACTTTGTCAAGCTCA-3′ (SEQ ID NO: 1) and 5′-GGTTGAGCACAGGGTACTTTATT-3′ (SEQ ID NO: 2) for GAPDH; and 5′-GAGTCCAGGTAAGTGAATCTGTCC-3′ (SEQ ID NO: 3) and 5′-ATTTCCACCGAGACCTCTCATC-3′ (SEQ ID NO: 4) for GALNT6 (GenBank #NM_{—}007210).

Breast cancer-northern blot membrane was prepared as previously described (Park, J. H., Lin, M. L., Nishidate, T., Nakamura, Y. & Katagiri, Cancer Res. 66, 9186-9195 (2006)), and hybridized with [alpha-³²P]-dCTP labeled PCR products of GALNT6 with the megaprime DNA labeling system (GE Healthcare, Buckinghamshire, UK). Pre-hybridization, hybridization and washing were performed as described previously (Katagiri, T. et al. Cytogenet. Cell Genet. 74, 90-95 (1996).). The blots were auto-radiographed with intensifying screens at −80 degrees C. for 14 days.

Constructs:

An open reading frame sequence of GALNT6 was obtained by RT-PCR using KOD-Plus DNA polymerase (Toyobo, Osaka, Japan) with the following primer sets:

(SEQ ID NO: 5)
5′-CGGAATTCATGAGGCTCCTCCGCAG-3′
and
(SEQ ID NO: 6)
5′-CCGCTCGAGGACAAAGAGCCACAACTGATG-3′
(underlines indicate recognition sites
of restriction enzymes).

The PCR product was inserted into the EcoRI and XhoI sites of pCAGGS-nHAc expression vector in frame with a HA-tag at the C-terminus. For construction of the GALNT6 enzyme-dead mutants, which contained a substitution at His271 (H271D) or Glu382 (E382Q) which corresponds to the residues that are reported to be essential to preserve the enzyme activity of GALNT1(Hagen, F. K., Hazes, B., Raffo, R., deSa, D. & Tabak, L. A. J. Biol. Chem. 274, 6797-6803 (1999)), a two-step mutagenesis PCR was performed (Park, J. H., Lin, M. L., Nishidate, T., Nakamura, Y. & Katagiri, T. Cancer Res. 66, 9186-9195 (2006)), using the following primer sets:

(SEQ ID NO: 7)
5′-GCTCACGTTCCTGGATGCCGACTGTGAGTGCTTCCACGG-3′
and
(SEQ ID NO: 8)
5′-CCGTGGAAGCACTCACAGTCGGCATCCAGGAACGTGAGC-3′
for H271D;
(SEQ ID NO: 9)
5′-CAGATGGAGATCTGGGGAGGGCAGAACGTGGAAATGTCCTTC-3′
and
(SEQ ID NO: 10)
5′-GAAGGACATTTCCACGTTCTG CCCTCCCCA-GATCTCCATCTG-3′
for E382Q
(underlines indicate nucleotides that were
replaced from the wild-type).

All of sequences were validated by DNA sequencing (ABI3700, PE Applied Biosystems, Foster, Calif.).

Generation of Anti-GALNT6 Specific Antibodies:

To generate anti-GALNT6 polyclonal antibodies, the partial coding sequence of GALNT6 protein (codons 35-179) was amplified by RT-PCR as mentioned above, using the following primer sets:

(SEQ ID NO: 11)
5′-CCGGAATTCGAGGAGGCCACAGAGAAGCC-3′
and
(SEQ ID NO: 12)
5′-CCGCTCGAGGGTGGTGGCCAGTGGGGGGC-3′
(underlines indicate recognition sites of
restriction enzymes).

The PCR products were cloned into the EcoRI and XhoI sites of pET28 vector (Novagen, Madison, Wis.) in frame with a His-tag in the C-terminus. The partial recombinant GALNT6 protein was expressed, purified and inoculated into rabbits, as described previously (Ueki, T. et al. Oncogene 27, 5672-5683 (2008). In addition, because of the limited amount of above polyclonal antibody, mouse anti-GALNT6 monoclonal antibodies were also generated. The partial recombinant GALNT6 protein (codons 35-622) was prepared as described in the following “Recombinant GALNT6 protein” section. After immunization into BALB/c mice, the lymph node cells were harvested and fused with the myeloma cell line as described previously (Fukukawa, C. et al. Cancer Sci. 99, 432-440 (2008)). The hybridomas were subcloned, and assayed by Western blot and immunocytochemical staining to assess the ability to recognize GALNT6 protein. After limiting dilution, the clones of #3G7 and #4H11 were selected for Western blot and immunostaining analyses, respectively. Finally, it was confirmed that the clones of #4H11 monoclonal antibody could specifically recognize endogenous GALNT6 protein in cell-lines by Western blot analysis without producing any non-specific bands, and the clones of #3G7 monoclonal antibody could specifically recognize endogenous GALNT6 protein by immunocytochemical staining without producing any background signals.

Recombinant GALNT6 Protein:

The partial coding sequence of GALNT6 without a signal peptide (codons 35-622) was amplified by PCR using the following primer sets;

(SEQ ID NO: 13)
5′-ATAAGAATGCGGCCGCAGAGGAGGCCACAGAGAAGCC-3′
and
(SEQ ID NO: 14)
5′-CGCGGATCCGACAAAGAGCCACAACTGATG-3′
(underlines indicate recognition sites of
restriction enzymes).

The PCR product was cloned into the NotI and BamHI sites of pQCXIPG-His expression vector (Medical and Biological Laboratories, Nagoya, Japan) in frame between a signal sequence peptide of immunoglobulin kappa chain (METDTLLLWVLLLWVPGSTG) (SEQ ID NO: 15) in the N-terminus and a His-tag in the C-terminus. The pQCXIPG-GALNT6-His vector was transfected into HEK293 cells using FuGENE6 transfection reagent (Roche, Basel, Switzerland), and then incubated in the approximate culture medium containing 2.0 microgram mL⁻¹ puromycin (Invitrogen, Carlsbad, Calif.). After selection for two weeks, cells were incubated in the medium without Fetal Bovine Serum (FBS) for 24 hours, and then culture media containing the secreted GALNT6 protein were collected. Subsequently, the recombinant His-tagged GALNT6 protein was purified by using Ni-NTA agarose (Qiagen, Valencia, Calif.) according to the supplier's protocol.

Immunocytochemical Staining:

The cells at 5×10⁴ were seeded in a 35-mm dish with a col-I coated glass (Iwaki, Tokyo, Japan) to examine the subcellular localization of endogenous GALNT6 protein in breast cancer cell-lines, T47D and MCF7. MCF10A cells were also prepared to examine the cellular-effects of the exogenously expressed GALNT6 protein. Forty-eight hours after incubation, cells were fixed with 4% paraformaldehyde in PBS (−) for 15 min, and rendered permeable with 0.1% Triton X-100 in PBS (−) at 4 degrees C. for 2.5 min. Subsequently, the cells were covered with 3% BSA in PBS (−) at 4 degrees C. for 3 hours to block non-specific hybridization followed by incubation with anti-GALNT6 polyclonal (diluted at 1:100) or anti-GALNT6 monoclonal antibodies (#3G7, diluted at 1:300). After washing with PBS (−) three times, the cells were stained by Alexa488-conjugated anti-rabbit or Alexa594-conjugated anti-mouse secondary antibodies (Molecular Probe, Eugene, Oreg.) diluted at 1:1000. Finally, nuclei were counter-stained with 4′, 6′-diamidine-2′-phenylindole dihydrochloride (DAPI) and fluorescent images were obtained under a TCS SP2 AOBS microscope (Leica, Tokyo, Japan). The Golgi apparatus and cytoskeleton structure were visualized by staining with anti Golgi-58k monoclonal antibody (Sigma-Aldrich, St. Louis, Mo.) and Alexa Fluor-594 phalloidin (Molecular Probe), respectively.

Immunohistochemical Staining:

Slides of paraffin-embedded breast cancer and normal specimens were stained with anti-GALNT6 polyclonal (diluted at 1:30), anti-GALNT6 monoclonal (#3G7, diluted at 1:40) and anti-MUC1 (#VU4H5, diluted at 1:50; Santa Cruz Biotechnology, Santa Cruz, Calif.) monoclonal antibodies, respectively, as described previously (Park, J. H., Lin, M. L., Nishidate, T., Nakamura, Y. & Katagiri, T. Cancer Res. 66, 9186-9195 (2006), Ueki, T. et al. Oncogene 27, 5672-5683 (2008)). For Immunohistochemical staining in normal human organs, the present inventors purchased tissue sections of heart, lung, liver and kidney from BioChain Institute Inc. (Hayward, Calif.).

Western Blot Analysis:

To detect the expression of endogenous GALNT6 and MUC1 proteins in breast cancer cells, cells were lysed with NP-40 lysis buffer (50 mM Tris-HCl/pH 8.0/150 mM NaCl/0.5% NP-40) including 0.1% protease inhibitor cocktail III (Calbiochem, San Diego, Calif.). After homogenization, the cell lysates were incubated on ice for 30 min and centrifuged at 18,000×g for 15 min to collect only supernatant. After quantification of total protein by a protein assay kit (Bio-Rad, Hercules, Calif.), each sample was mixed with SDS-sample buffer and boiled before loading at SDS-PAGE gel. After electrophoresis, the proteins were transferred onto nitrocellulose membrane (GE Healthcare). Membranes blotted with proteins were blocked by 4% BlockAce solution (Dainippon Pharmaceutical, Osaka, Japan) for overnight, and incubated with an anti-GALNT6 polyclonal antibody or anti-GALNT6 (#4H11), anti-MUC1 (#VU4H5), anti-beta-catenin (#E-5, Santa Cruz Biotechnology), anti-E-cadherin (BD Biosciences, San Jose, Calif.), and anti-beta-actin (Sigma-Aldrich) monoclonal antibodies. Particularly, the anti-MUC1 monoclonal antibody (#VU4H5) can recognize the endogenous MUC1 proteins at various molecular weights, which are explainable by its structural features of the gene containing VNTR (variable numbers of tandem repeat) and the protein forming homo- or hetero-dimers (Gendler, S. J. et al. J. Biol. Chem. 265, 15286-15293 (1990), Thathiah, A., Blobel, C. P. & Carson, D. D. J. Biol. Chem. 278, 3386-3394 (2003)). Finally the membrane was incubated with HRP conjugated secondary antibodies (Santa Cruz Biotechnology), and the protein bands were visualized by ECL detection reagents (GE Healthcare).

VVA-Lectin Blot and Pull Down:

To detect the GalNAc-conjugated proteins, lectin western blot was conducted as described previously (Qiu, Y. et al. J. Proteome Res. 7, 1693-1703 (2008)). Briefly, whole cell lysates including glycoproteins of interest was transferred onto a nitro-cellulose membrane (GE Healthcare) after SDS-PAGE. The membrane was blocked by 5% BSA in TBST at 4 degrees C. for overnight, followed by incubation with 0.5 microgram mL⁻¹ of biotin conjugated VVA lectin (EY laboratories, San Mateo, Calif.) in TBST containing 3% BSA for 1 hour. The membrane was then washed, and incubated for 30 min with Streptavidin HRP (BD Biosciences), diluted at 1:20,000. After washing three times, the signal was visualized by the ECL detection reagents (GE Healthcare). Similarly, the proteins bound to the biotin-VVA lectin were pulled down by Streptavidin agarose (Invitrogen) as described previously (Seales, E. C., Jurado, G. A., Singhal, A. & Bellis, S. L. Oncogene 22, 7137-7145 (2003)).

Gene-Silencing by RNA Interference (RNAi):

To knock down endogenous GALNT6 expression in breast cancer cells, the psiU6BX3.0 vector was used for expression of short hairpin RNA (shRNA) against a target gene as describe previously (Shimokawa, T. et al. Cancer Res. 63, 6116-6120 (2003)). Target sequences of the synthetic oligonucleotides for shRNA against GALNT6 were shown in Table-1. Each of shRNA expression vector were transfected into T47D cells using FuGENE6 transfection regent (Roche) according to the supplier's recommendations. Ten days after transfection, to evaluate the knockdown effect on GALNT6 expression, semiquantitative RT-PCR was performed using the primer set as describe above. The cell viability was quantified by MTT and colony formation assays, as described previously (Park, J. H., Lin, M. L., Nishidate, T., Nakamura, Y. & Katagiri, T. Cancer Res. 66, 9186-9195 (2006)). To examine the early stage effects in the cells in which GALNT6 or MUC1 was knocked down, the synthesized duplex siRNAs (Sigma Aldrich Japan KK, Tokyo, Japan); si-EGFP (5′-GCAGCACGACUUCUUCAAG-3′) (SEQ ID NO: 16) and si-GALNT6 (5′-GAGAAAUCCUUCGGUGACA-3′) (SEQ ID NO: 17) corresponding to the target sequence of sh-G6-2 were also used. The si-MUC1 (5′-GUUCAGUGCCCAGCUCUAC-3′) (SEQ ID NO: 18) was synthesized according to the previous report (Ren, J. et al. Cancer Cell 5, 163-175 (2004)). Breast cancer cells (MCF-7, T47D and SKBR-3) were plated onto 6-cm dishes (2×10⁵ cells/dish) and transfected with 100 pmol each of the synthesized duplex siRNAs using a Lipofectamine RNAiMAX reagent (Invitrogen), according to the manufacturer's instructions. Four days later, knockdown of target proteins and corresponding cell morphology were monitored by Western blot and immunocytochemistry.

TABLE 1
Sequences of double-strand oligonucleotides inserted into shRNA expression vector

psi-U6BX-Mock (control)
5′-CACCGTGTCTTCAAGCTTGAAGACTA-3′
(SEQ ID NO: 19)
5′-AAAATAGTCTTCAAGCTTGAAGACAC-3′
(SEQ ID NO: 20)
psi-U6BX-sh-EGFP (control)
5′-CACCGAAGCAGCACGACTTCTTCTTCAAGAGAGAAGAAGTCGTGCTGCTTC-3′
(the sequence of double-strand oligonucleotide inserted into shRNA expression vector
targeting SEQ ID NO: 21)
5′-AAAAGAAGCAGCACGACTTCTTCTCTCTTGAAGAAGAAGTCGTGCTGCTTC-3′
(the sequence of double-strand oligonucleotide inserted into shRNA expression vector
targeting SEQ ID NO: 21)
psi-U6BX-sh-G6-1
5′-CACCGCACTGTTTCAATGCCTTTTTCAAGAGAAAAGGCATTGAAACAGTGC-3′
(the sequence of double-strand oligonucleotide inserted into shRNA expression vector
targeting SEQ ID NO: 22)
5′-AAAAGCACTGTTTCAATGCCTTTTCTCTTGAAAAAGGCATTGAAACAGTGC-3′
(the sequence of double-strand oligonucleotide inserted into shRNA expression vector
targeting SEQ ID NO: 22)
psi-U6BX-sh-G6-2
5′-CACCGAGAAATCCTTCGGTGACATTCAAGAGATGTCACCGAAGGATTTCTC-3′
(the sequence of double-strand oligonucleotide inserted into shRNA expression vector
targeting SEQ ID NO: 23)
5′-AAAAGAGAAATCCTTCGGTGACATCTCTTGAATGTCACCGAAGGATTTCTC-3′
(the sequence of double-strand oligonucleotide inserted into shRNA expression vector
targeting SEQ ID NO: 23)
psi-U6BX-sh-mis-1*
5′-CACCCAGAATTCCATCGGTGACTTTCAAGAGAAGTCACCGATGGAATTCTG-3′
(the sequence of double-strand oligonucleotide inserted into shRNA expression vector
targeting SEQ ID NO: 24)
5′-AAAACAGAATTCCATCGGTGACTTCTCTTGAAAGTCACCGATGGAATTCTG-3′
(the sequence of double-strand oligonucleotide inserted into shRNA expression vector
targeting SEQ ID NO: 24)
psi-U6BX-sh-mis-2*
5′-CACCCAGAACTCCATCGGTGACTTTCAAGAGAAGTCACCGATGGAGTTCTG-3′
(the sequence of double-strand oligonucleotide inserted into shRNA expression vector
targeting SEQ ID NO: 25)
5′-AAAACAGAACTCCATCGGTGACTTCTCTTGAAAGTCACCGATGGAGTTCTG-3′
(the sequence of double-strand oligonucleotide inserted into shRNA expression vector
targeting SEQ ID NO: 25)
*mismatched oligonucleotides were designed from sh-G6-2 by substitution of some internal bases

Cell Detachment Assay:

The strength of cell to culture dish attachment was quantified by the “cell detachment assay (Gordon, P. B., Levitt, M. A., Jenkins, C. S. & Hatcher, V. B. J. Cell Physiol. 121, 467-475 (1984), Uzdensky, A., Kolpakova, E., Juzeniene, A., Juzenas, P. & Moan, J. Biochim. Biophys. Acta 1722, 43-50 (2005)). Briefly, T47D cells were seeded at 1×10⁵ cells per well in 6-well plate dishes (in triplicate) after transfection with si-EGFP, si-GALNT6 or si-MUC1 (see above). Four days later, total number of viable cells was evaluated by MTT assay (1st MTT) using Cell Counting Kit-8 (Dojindo, Kumamoto, Japan). Then, the MTT reagent was removed by washing with PBS (−), and the cells were incubated with a dissociation solution containing 5 mM of EDTA in PBS (−) for 10 min. The dissociated cells were removed by washing with PBS (−), and incubated with fresh culture medium for 12 hours. Subsequently, total number of the remained cells was estimated by MTT assay (2^{nd} MTT). The strength of cell attachment was calculated by the percentage of remained cells (2^{nd} MTT) to starting cells (1^{st} MTT).

Establishment of GALNT6-Stably Expressed-Transformants:

Mock (no insert) or pCAGGS-GALNT6 (WT and H271D)-HA expression vectors were transfected into HeLa cells using FuGENE6 transfection reagent (Roche). Then, the positive clones were selected under incubation with culture medium containing 0.8 mg mL⁻¹ of neomycin (Geneticin, Invitrogen). Two weeks later, the stable trans-formants were selected by the limiting dilution, and screened for clones stably expressing HA-tagged GALNT6 protein (WT and H271D). Finally, individual clones of mock (#001, 003, and 006), WT (#101, 110, and 304), and H271D (#102, 212, and 114) were isolated.

In vitro GalNAc-Transferase Assay:

The partial length of recombinant GALNT6 protein without a signal peptide (35-622 amino-acid) was purified from HEK293T stable transformants (see above). As substrates, MUC1-a (AHGVTSAPDTR) (SEQ ID NO: 26) and MUC1-b (RPAPGSTAPPA) (SEQ ID NO: 27) peptides derived form the tandem repeat of MUC1 protein were synthesized by Sigma-Aldrich Japan (Tokyo, Japan) and fluorescence-labeled (dansylation, DNS) as reported previously (Takegawa, K. et al. J. Biol. Chem. 270, 3094-3099 (1995), Bennett, E. P. et al. J. Biol. Chem. 274, 25362-25370 (1999)). For in vitro GalNAc-transferase assay, reaction was performed in 50 microliter of reaction mixtures containing 25 mM Tris-HCl (pH 7.4), 10 mM MnCl₂, 50 microM UDP-GalNAc, 4 microM DNS-MUC1 peptides, and 0.5 microgram of recombinant GALNT6 protein. The reaction mixture was incubated at 37 degrees C. for 10 min to 16 hours and stopped by heating at 100 degrees C. for 2 min. Finally, the reaction mixture was centrifuged, and the supernatant was analyzed by HPLC with a Wakosil 5C18 column (4.6×250 mm) (Wako, Osaka, Japan), equilibrated with 0.1% trifluoroacetic acid, and eluted with a linear acetonitrile gradient (40% in 40 min) at a flow rate of 1.0 mL min⁻¹. The products were detected by fluorescence (excitation wavelength, 313 nm; emission wavelength, 540 nm). For the confirmation of GalNAc-conjugation, the reacted samples were further incubated with Acremonium sp. alpha-N-acetylgalactosaminidase (GalNAcase) (Seikagaku Biobusiness, Tokyo, Japan) to remove the conjugated GalNAc from the MUC1-a peptide.

Statistical Analysis:

Statistical significance was calculated by Student's t-test using Statview 5.0 software (SAS Institute). A difference of p<0.05 was considered to be statistically significant.

### Example 2

### Identification of GALNT6 Upregulated in Breast Cancer

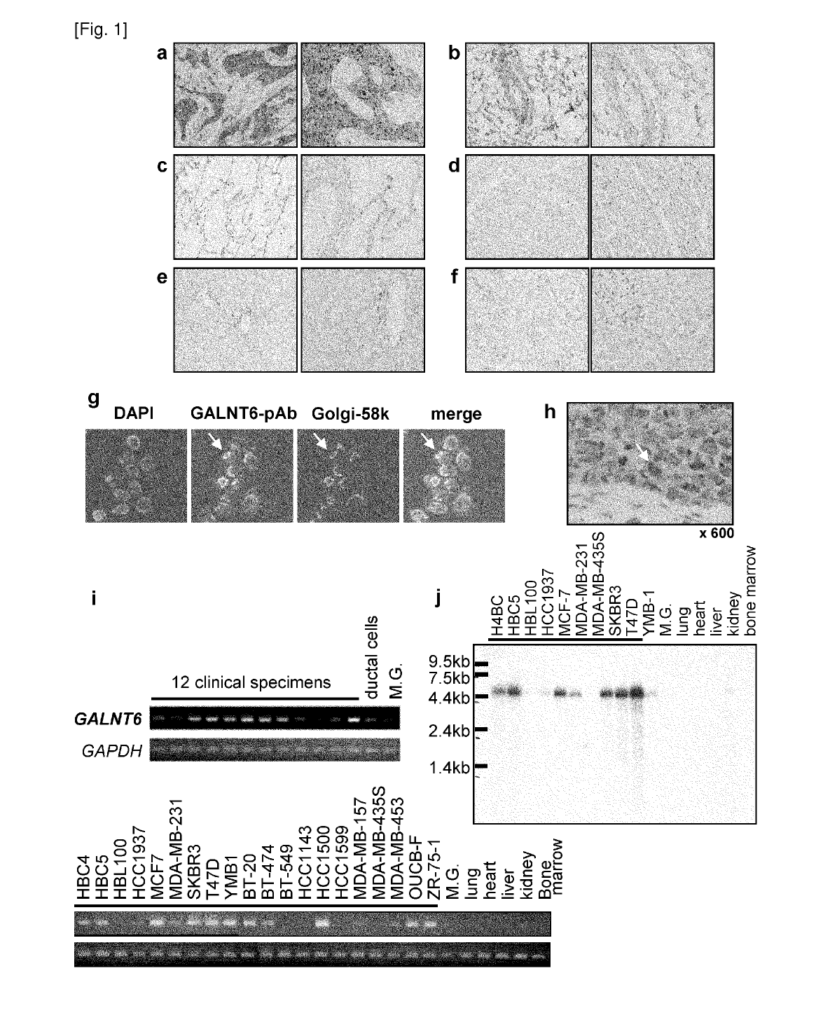

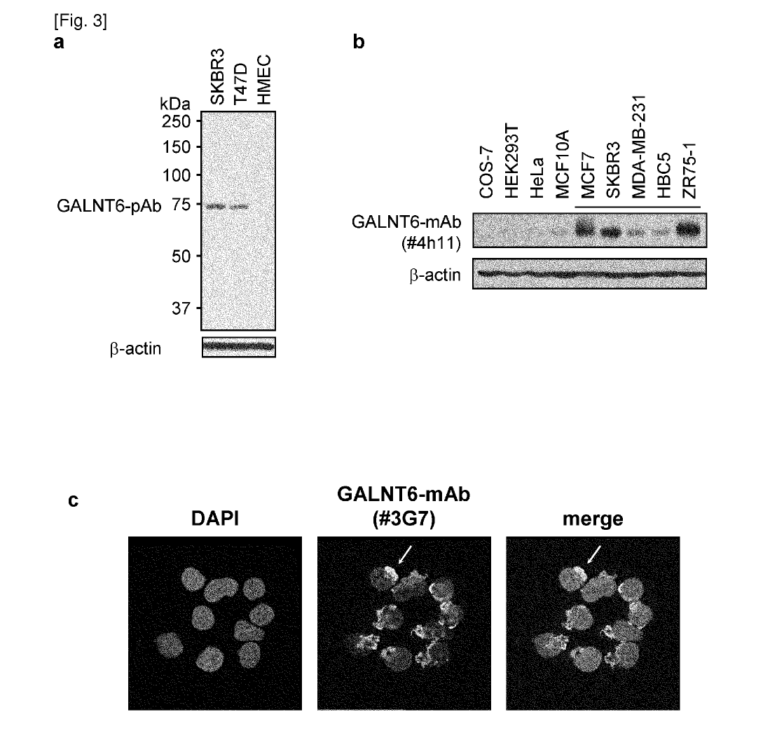

Through the previous studies of genome-wide gene expression profiles (Nishidate, T. et al. Int. J. Oncol. 25, 797-819 (2004), Saito-Hisaminato, A. et al. DNA Res. 9, 35-45 (2002)), genes that encode proteins having enzymatic activity, according to the reported information or the computer-assisted prediction by SMART program (http://smart.embl-heidelberg.de), have been identified and selected for further study. One in particular—the GALNT6 gene that encodes an O-glycosyltransferase—is a putative drug target for breast cancer. Its up-regulation in 7 of 12 clinical breast cancer specimens, and 12 of 19 breast cancer cell lines examined was confirmed by semiquantitative RT-PCR analysis (FIG. 1i). Subsequent northern blot analysis revealed overexpression of its approximately 5-kb transcript in breast cancer cell lines, while its expression was hardly detectable in normal human organs (FIG. 1j) as concordant to the results of cDNA microarray analysis. Rabbit polyclonal and two kinds of mouse monoclonal antibodies (#3G7 and #4H11) were subsequently generated, all of which could recognize the endogenous GALNT6 protein (˜75 kDa) in breast cancer cells without producing any non-specific bands or background signals in SDS-PAGE and immunocytochemical staining (FIG. 3a-c). The immunohistochemical staining analysis revealed its strong staining in breast cancer tissues (FIG. 1a), whereas no positive staining in the normal human tissues including normal mammary ductal cells, lung, heart, liver and kidney (FIG. 1b-f), in concordance with the results of northern blot analysis.

To further characterize the GALNT6 protein in breast cancer cells, the subcellular-localization of endogenous GALNT6 protein was investigated in T47D breast cancer cells by immunocytochemical staining using anti-GALNT6 polyclonal antibody. The results showed that GALNT6 protein was clearly observed in the Golgi complex of T47D cells, as evaluated by co-staining with Golgi marker, Golgi-58k (FIG. 1g). Similarly, it was observed strong staining of GALNT6 protein in the Golgi complex in breast cancer tissue section (FIG. 1h).

### Example 3

### Knockdown of GALNT6

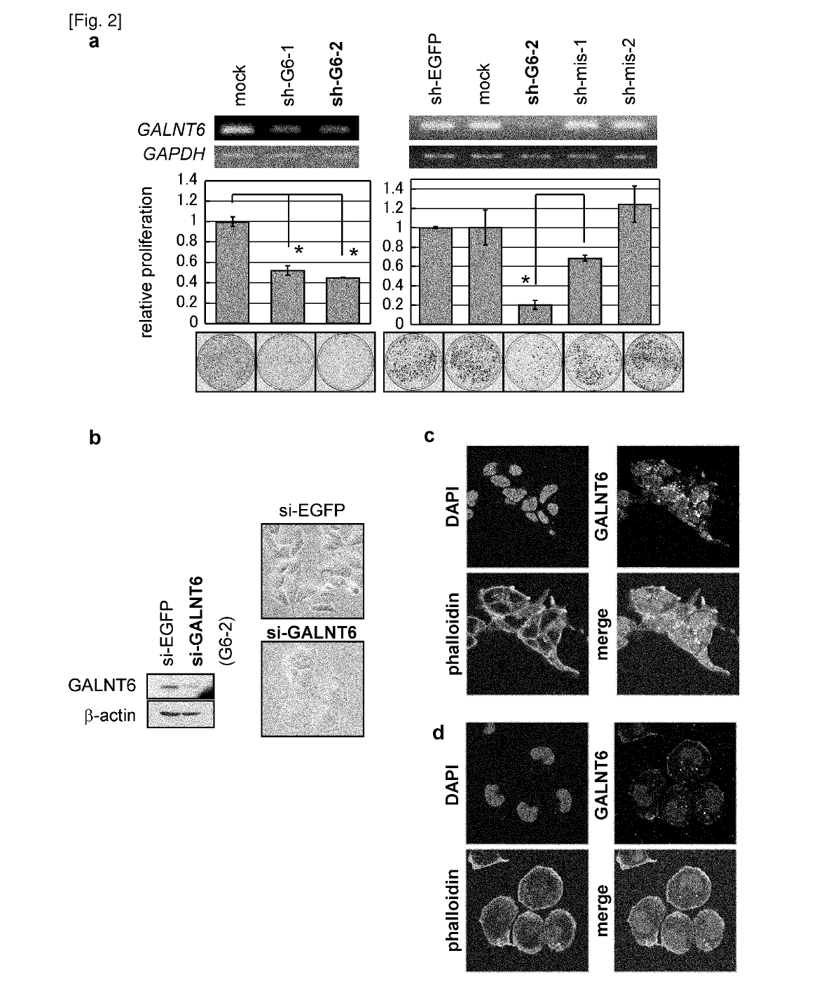

To investigate the biological significance of GALNT6 overexpression in breast cancer cells, short hairpin RNA (shRNA) expression vectors were generated to knockdown the endogenous expression of GALNT6 (sh-G6-1 and sh-G6-2). It was discovered that introduction of both of sh-G6-1 and -2 into T47D cells resulted in significant reduction of GALNT6 expression that was accompanied by suppression of cell proliferation, while no change was observed in the cells transfected with a control shRNA vector (FIG. 2a, left). Moreover, the results of sh-G6-2 specificity to GALNT6 were confirmed by using two mismatched shRNAs (sh-mis-1 and -2) (FIG. 2a, right). To further examine the effects of GALNT6 knockdown, the synthesized oligo-duplex siRNA against GALNT6 (si-GALNT6) was introduced into T47D cells. Interestingly, four days after the transfection of siRNA, the GALNT6-depleted (si-GALNT6) cells showed round shape and enlarged cell size, compared with the cells transfected with a control si-EGFP (FIG. 2b). These morphologic alterations caused by si-GALNT6 were further assessed by immunostaining with fluorescence-labelled phalloidin to clarify the cell shape (FIG. 2c, d).

### Example 4

### Stabilization of MUC1 by GALNT6

It was observed that the appearances of GALNT6-depleted cells were very similar to those of the cells in which MUC1 was knocked down (Wesseling, J., van der Valk, S. W., Vos, H. L., Sonnenberg, A., Hilkens, J. J. Cell Biol. 129, 255-265 (1995), Yuan, Z., Wong, S., Borrelli, A. & Chung, M. A. Biochem. Biophys. Res. Commun. 362, 740-746 (2007)). Since MUC1 was reported to be one of candidate substrates of the GALNT family (Bennett, E. P. et al. J. Biol. Chem. 274, 25362-25370 (1999)), the knockdown effects of GALNT6 were compared with that of MUC1 by siRNA using breast cancer cell-lines, T47D, MCF7, and SKBR3. First, as shown in FIG. 5a, it was confirmed the knockdown of GALNT6 and MUC1 expressions by western-blot analysis using an anti-GALNT6 monoclonal antibody (#3G7) and anti-MUC1 monoclonal antibody (clone #VU4H5) that could specifically recognize endogenous MUC1 in breast caner cells (data not shown). As expected, either of GALNT6- or MUC1-depletion caused the very similar morphologic changes (round shape and enlarged cell size) and the attenuated cell proliferation in all the three cell lines examined (FIG. 5b, c). These findings have indicated that GALNT6 is likely to be indispensable for the proliferation of breast cancer cells through the regulation of cytoskeleton structure possibly by modification of MUC1.

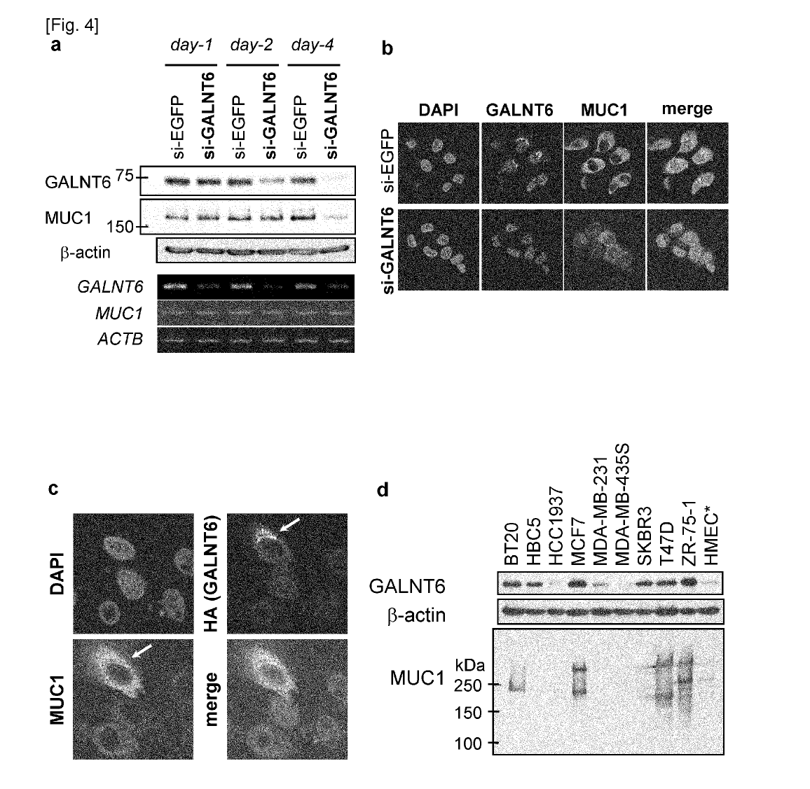

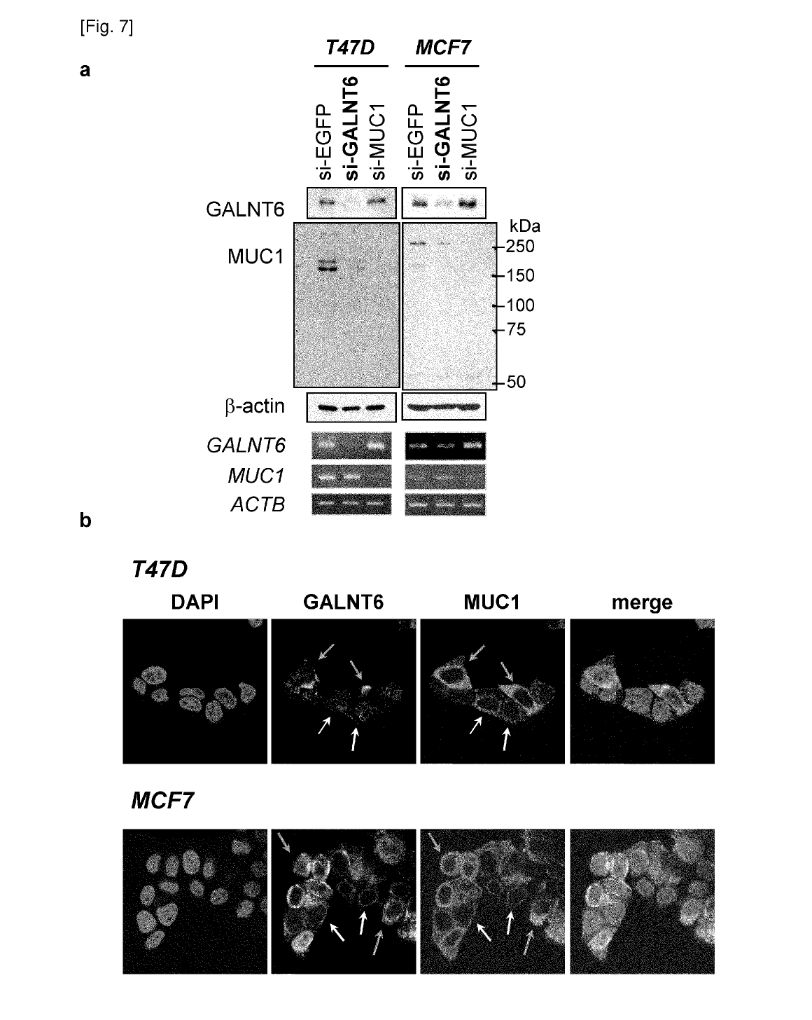

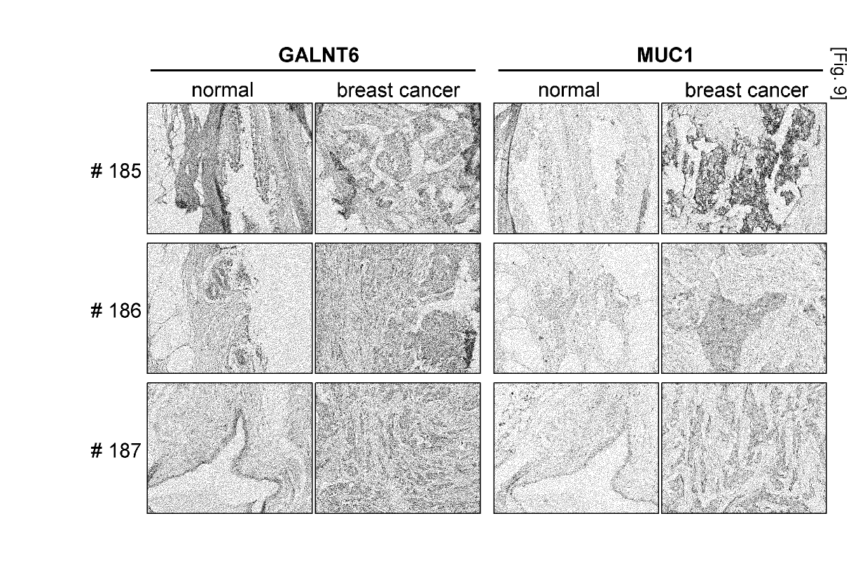

To investigate the interaction of GALNT6 and MUC1 in more detail, GALNT6 expression was knocked down by siRNA and its effect on the MUC1 protein was examined in T47D cells. It was discovered that knockdown of GALNT6 protein induced the reduction of cytoplasmic MUC1 protein (4 days after the transfection; FIG. 4a, b), although the transcriptional level of MUC1 was unchanged (FIG. 4a). When another cell line, MCF7 was used, the similar results were observed (FIG. 7a, b), suggesting that GALNT6 may influence the posttranslational modification and stabilization of MUC1 protein in breast cancer cells. The plasmid designed to express GALNT6 protein was subsequently introduced into MCF10A cells, in which the GALNT6 expression level was very low (FIG. 3b). Immunocytochemical staining demonstrated the remarkable enhancement of the signal intensity of cytoplasmic MUC1 protein by introduction of GALNT6 (FIG. 4c, arrow), further supporting the hypothesis that GALNT6 plays a critical role for the stabilization of MUC1 protein. In addition, the expression levels of these two molecules in breast cancer cells were examined by Western blot analysis with anti-GALNT6 and anti-MUC1 monoclonal antibodies, and found that GALNT6 and MUC1 proteins were co-overexpressed in breast cancer cell lines and clinical cancer tissue sections examined, but neither of the proteins was expressed in HMEC or normal breast ductal cells (FIG. 4d, FIG. 9). Taken together, these findings imply that upregulation of GALNT6 protein contributes to mammary carcinogenesis through stabilization of MUC1 oncoprotein.

### Example 5

### GALNT6 O-glycosylates MUC1 In Vitro and In Vivo

To investigate whether GALNT6 O-glycosylates MUC1 as a substrate, the recombinant wild-type GALNT6 (WT) and the inactive GALNT6 mutant proteins (H271D and E382Q) were generated, and in vitro GalNAc-transferase assays were performed using MUC1 peptides (MUC1-a and -b) corresponding to the tandem repeat fragment of MUC1 protein. WT-GALNT6 rapidly O-glycosylated MUC1 peptides in 10-min incubation as indicated by the left-shifted band in FIG. 6a. On the other hand, mutant-GALNT6 proteins (H271D and E382Q) could not transfer GalNAc to MUC1 peptides even by 16 hours incubation (no left-shifted band appeared) (FIG. 6b). In addition, it was confirmed that treatment of GalNAcase removed GalNAc, which was transferred by the WT-GALNT6, and restored the left-shifted peak of MUC1-a peptide (FIG. 6c).

To further investigate whether the exogenous introduction of GALNT6 protein can glycosylate the endogenous MUC1 protein in vivo, Western blot analysis was performed with anti-MUC1 monoclonal antibody using the HeLa-derivative cells in which stable GALNT6 expression was established; those in which mock or H271D expression vectors were introduced were used as controls (mock, WT, and H271D; see Materials and Methods). In the cells with WT-GALNT6 (clone no. 101, 110 and 304), the highest molecular weight of MUC1 protein (>250 kDa) was observed, while in those with mock (clone no. 001, 003 and 006) or H271D (clone no. 102, 212 and 114) the shifted band was not observed (FIG. 6d). Moreover, the shifted MUC1 protein corresponded to the O-glycosylated (GalNAc) MUC1 protein was confirmed by immunoprecipitation with anti-MUC1 monoclonal antibody followed by VVA-lectin blotting (FIG. 6e) and vice versa (FIG. 6f). To further examine whether GALNT6 stabilizes the MUC1 protein in vivo, WT or H271D constructs were transfected into MCF10A normal epithelial cells, and then performed immunocytochemical staining with anti-HA- and anti-MUC1 antibodies (FIG. 6g). The WT-GALNT6 transformants augmented the signal intensity of MUC1 proteins (arrows in upper panels), whereas the H271D did not affect that of MUC1 (arrows in lower panels), suggesting that GALNT6-mediated glycosylation of MUC1 protein is critical for stability of MUC1 protein.

### Example 6

### GALNT6 and MUC1 are Involved in Cytoskeletal Regulation

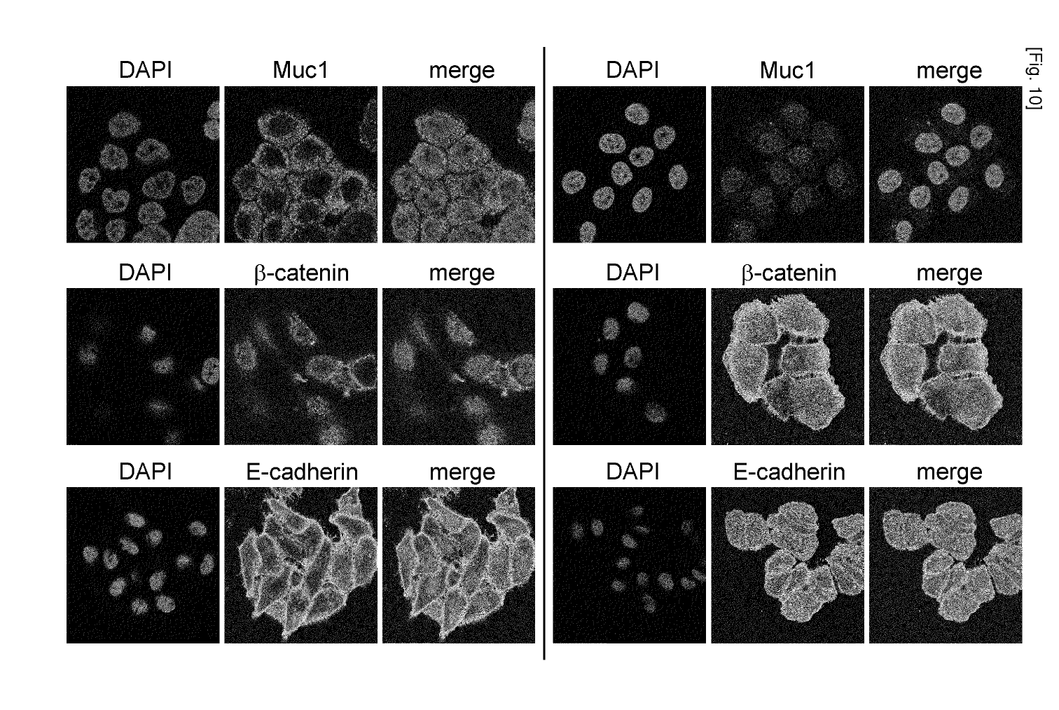

Since MUC1 was reported to disrupt cell adhesion (Yuan, Z., Wong, S., Borrelli, A. & Chung, M. A. Biochem. Biophys. Res. Commun. 362, 740-746 (2007), Schroeder, J. A., Adriance, M. C., Thompson, M. C., Camenisch, T. D. & Gendler, S. J. Oncogene 22, 1324-1332 (2003)), the participation of two cell adhesion molecules, beta-catenin and E-cadherin, which were reported their involvement in carcinogenesis, in the GALNT6-MUC1 pathway, were examined. GALNT6 and MUC1 expressions were knocked down by siRNA in T47D breast cancer cells. The results of semiquantitative RT-PCR and Western blot analyses confirmed that knockdown of either GALNT6 or MUC1 remarkably enhanced the amount of these cell adhesion molecules at protein level (FIG. 8a, upper panels), but did not alter their transcriptional levels (FIG. 8a, lower panels). T47D cells were immunostained with or without GALNT6-knockdown using anti-beta or E-cadherin monoclonal antibodies (FIG. 8b, c), and cell morphologic changes (round shape and enlarged size) accompanied by stronger staining of beta-catenin (FIG. 8b) and E-cadherin (FIG. 8c) proteins were identified. The results of MUC1-depleted T47D cells were quite similar to those of GALNT6-depleted cells (FIG. 10). Since the increase of the cell adhesion complex might enhance cell-to-plate dish attachment, the “cell detachment assay” (see Materials and Methods) was performed and found the inverse correlation between MUC1 expression level and strength of the cell attachment (FIG. 11).

### Discussion

Among all human genes, approximately 2000-3000 genes are estimated to encode drug proteins, which include membrane or nuclear receptors, ion channels, protein kinases and other enzymes (Clarke, P. A., to Poele, R. & Workman, P. Eur. J. Cancer 40, 2560-2591 (2004)). The comparison of whole-genome expression profiles between a large set of normal and cancer cells has been considered to be an effective approach to identify potential targets for development of anti-cancer drugs (Stoughton, R. B. & Friend, S. H. Nature Rev. Drug Discov. 4, 345-350 (2005)).

Since the reduction of adverse reactions caused by drugs, particularly by anti-cancer agents, is one of the very serious issues to be solved in clinical management, the present invention focused on the isolation of cancer-specific molecules that were upregulated commonly in cancer cells, but were not or undetectably expressed in normal human organs (Nishidate, T. et al. Int. J. Oncol. 25, 797-819 (2004), Saito-Hisaminato, A. et al. DNA Res. 9, 35-45 (2002)). A number of cancer-specific molecules have been identified and characterized for possible application to development of cancer therapy (Park, J. H., Lin, M. L., Nishidate, T., Nakamura, Y. & Katagiri, T. Cancer Res. 66, 9186-9195 (2006), Lin, M. L., Park, J. H., Nishidate, T., Nakamura, Y. & Katagiri, T. Breast Cancer Res. 9, R17 (2007), Kanehira, M. et al. Cancer Res. 67, 3276-3285 (2007), Fukukawa, C. et al. Cancer Sci. 99, 432-440 (2008)).

In the context of the present invention, a novel breast cancer-specific molecule, GALNT6, encoding an O-glycosyltransferase was characterized and, by showing its critical role in the growth of breast cancer cells, was demonstrated to have potential as a cancer drug target.

O-type glycosylation is one of many common modifications that have multiple functions related to the folding, stability, and targeting of various glycoproteins, and is initiated by members belonging to the GALNT family in the Golgi complex (Carraway, K. L. 3rd, Funes, M., Workman, H. C. & Sweeney, C. Curr. Top. Dev. Biol. 78, 1-22 (2007)). Accumulating evidence suggests that the GALNT family members are involved in several cellular functions by catalyzing substrates specific to each member. For instance, glycosylation by GALNT3 prevents proteolytic processing of FGF23 (fibroblast growth factor 23) and that by GALNT14 promotes ligand-stimulated clustering of death receptors (Wagner, K. W. et al. Nature Med. 13, 1070-1077 (2007), Ichikawa, S. et al. Endocrinology 150, 2543-2550 (2009)).

Abnormalities of the glycan structure of proteins are frequently observed in breast cancer cells (Brockhausen, I. EMBO Rep. 7, 599-604 (2006)). Immunostaining analysis in the present invention revealed very intense staining of GALNT6 in the Golgi apparatus of breast cancer cells, but no staining in adjacent normal cells, suggesting its potential roles in mammary carcinogenesis through protein glycosylation. Subsequent knockdown experiments of GALNT6 by siRNA detected mor-phologic alterations such as round shape and enlarged cell size as well as suppression of the growth of cancer cells. Hence, the GALNT6 overexpression in breast cancer cells was considered to be tightly linked to regulation of cytoskeleton structure and also proliferation of breast cancer cells. The morphologic alterations observed in GALNT6-depleted cells closely resembled those of MUC1-depleted cells (Wesseling, J., van der Valk, S. W., Vos, H. L., Sonnenberg, A., Hilkens, J. J. Cell Biol. 129, 255-265 (1995); Yuan, Z., Wong, S., Borrelli, A. & Chung, M. A. Biochem. Biophys. Res. Commun. 362, 740-746 (2007)). Since MUC1 is a well-known glycoprotein having an oncogenic function, a possible interaction of GALNT6 with MUC1 was considered. Subsequently, a reduction of MUC1 protein in the GALNT6-depleted cancer cells by siRNA was discovered through immunoblotting and immunostaining analyses. Because O-type glycosylation has been suggested as a means to regulate protein stability during recycling of MUC1 (Altschuler, Y. et al. Mol. Biol. Cell 11, 819-831 (2000)), it was hypothesized that activated glycosylation of MUC1 by GALNT6 could enhance MUC1 stability. Exogenous introduction of GALNT6 in MCF10A cells, in which the expression level of GALNT6 protein was very low, resulted in elevated immunostaining of MUC1 protein, further supporting the central hypothesis of the present invention. Additionally, these findings demonstrate that the GALNT6 and MUC1 proteins are frequently co-upregulated in breast cancer cells and clinical breast cancer tissues.

The MUC1 protein was then investigated as a candidate substrate for GALNT6 protein. An in vitro enzyme assay was first performed using MUC1 peptides, and it was demonstrated that wild-type-GALNT6 (WT-GALNT6) recombinant protein O-glycosylated MUC1 in vitro, but enzyme-dead GALNT6 mutants (H271D and E382Q) did not. Additionally, it the WT-GALNT6 and H271D-GALNT6-stably expressing cells were established, and used to demonstrate that the WT-GALNT6 protein induced O-glycosylation of MUC1 in vivo, but the H271D-GALNT6 mutant did not, suggesting that the GALNT6 protein can O-glycosylate MUC1 in vitro as well as in vivo.

To further characterize the biological significance of the GALNT6 and MUC1 interaction, the status of beta-catenin and E-cadherin were examined because these two molecules are known to be involved in carcinogenesis and also important in the regulation of cell morphology. According to previous reports, it is likely that MUC1 captures beta-catenin through interaction with its cytoplasmic tail and thereby inhibits complex formation of cell adhesion molecules (Yuan, Z., Wong, S., Borrelli, A. & Chung, M. A. Biochem. Biophys. Res. Commun. 362, 740-746 (2007), Schroeder, J. A., Adriance, M. C., Thompson, M. C., Camenisch, T. D. & Gendler, S. J. Oncogene 22, 1324-1332 (2003)). It was herein discovered that the GALNT6-MUC1 pathway plays a very significant role in stabilization and localization of these two molecules, and formation of the cell adhesion complex. To quantify the GALNT6/MUC1-mediated disruption of cell-adhesion, a cell detachment assay was performed. The results demonstrated that attachment (cell-to =dish) was clearly inhibited by accumulation of the MUC1 protein in a concordance with previous findings (Wesseling, J., van der Valk, S. W., Vos, H. L., Sonnenberg, A., Hilkens, J. J. Cell Biol. 129, 255-265 (1995)).

In summary, the findings of the present invention suggest a mechanism as described in FIG. 8d. In breast cancer cells, up-regulation of GALNT6 appears to stabilize the MUC1 protein throughout its glycosylation activity. Subsequently, the accumulation of glycosylated MUC1 protein appears to induce abnormalities of the cell adhesion molecules, such as beta-catenin and E-cadherin, thereby resulting in the anti-adhesive effect. In addition, accumulation of beta-catenin has been demonstrated to enhance the Tcf-signaling pathway. Experimental data in colon cancer cells has indicated that beta-catenin functions as an oncoprotein through its ability to interact with the Tcf/LEF transcriptional complex, translocates to the nucleus, and transactivates oncogenes such as c-myc, and cyclin D1 (He, T. C. et al. Science 281, 1509-1512 (1998), Shtutman, M. et al. Proc. Natl. Acad. Sci. USA 96, 5522-5527 (1999)). Moreover, the elevated MUC1 protein promotes cancer cell proliferation partly by interactions with EGFR, c-Src, Grb2, and ER-alpha (Singh, P. K. & Hollingsworth, M. A. Trends Cell Biol. 16, 467-476 (2006), Wei, X., Xu, H. & Kufe, D. Mol. Cell 21, 295-305 (2006)), although further depth analysis will be required to elucidate the precise mechanism of the GALNT6-MUC1 pathway in breast cancer cells.

### INDUSTRIAL APPLICABILITY

The data provided herein add to a comprehensive understanding of cancers, facilitate development of novel diagnostic strategies, and provide clues for identification of molecular targets for therapeutic drugs and preventative agents. Such information contributes to a more profound understanding of tumorigenesis, and provides indicators for developing novel strategies for diagnosis, treatment, and ultimately prevention of cancers.

In particular, the data herein confirm the critical roles of GALNT6 and MUC1 as drug targets in the diagnosis, treatment and prevention of cancer, particularly breast cancer. As noted above, GALNT6 is upregulated in a great majority of breast cancers and encodes a glycosyltransferase responsible of initiating mutin-type O-glycosylation in mammary carcinogenesis. Knockdown of GALNT6 and MUC1 by small-interfering RNA (siRNA) significantly enhanced cell adhesion function and suppressed the growth of breast cancer cells. Western-blot and immunocytochemical analyses indicated that wild-type GALNT6 protein could glycosylate and stabilize an oncoprotein MUC1.

Immunohistochemical staining analysis confirmed co-upregulation of GALNT6 and MUC1 proteins in breast cancer specimens. Furthermore, knockdown of GALNT6 or MUC1 led to similar morphologic changes (round shape and enlarged size) of cancer cells accompanied by the increase of cell adhesion molecules, beta-catenin and E-cadherin. Taken together, the data herein suggest that overexpression of GALNT6 may contribute to mammary carcinogenesis through aberrant glycosylation and stabilization of MUC1 protein and, thus, that the inhibitors of the enzymatic activity of GALNT6 protein may serve as valuable targets in the development of therapeutic modalities against breast cancer.

Thus, the present invention may contributes the development of novel cancer therapeutic strategy by providing the screening method for drag candidates using the interaction between GALNT6 protein and MUC1 protein as an index.

All patents, patent applications, and publications cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method of screening for a candidate substance for treating or preventing cancer or inhibiting the binding between a GALNT6 polypeptide and a MUC1 polypeptide, said method comprising the steps of:
(a) contacting a GALNT6 polypeptide or functional equivalent thereof with a MUC1 polypeptide or functional equivalent thereof, in the presence of a test substance;
(b) detecting the binding between the polypeptides; and
(c) selecting the test substance that inhibits the binding between the polypeptides.

2. The method of claim 1, wherein the functional equivalent of the GALNT6 polypeptide comprises a MUC1 binding domain of the GALNT6 polypeptide.

3. The method of claim 1, wherein the functional equivalent of the MUC1 polypeptide comprises a GALNT6 binding domain of the MUC1 polypeptide.

4. A method of screening for a candidate substance for treating or preventing cancer or inhibiting the glycosylation of a substrate by a GALNT6 polypeptide, said method comprising the steps of:
(a) incubating a GALNT6 polypeptide or functional equivalent thereof and a substrate in the presence of a test substance under a condition suitable for the glycosylation of the substrate by the GALNT6 polypeptide;
(b) detecting a substrate glycosylation level;
(c) comparing the substrate glycosylation level to a control level, wherein an increase or decrease in the glycosylation level as compared to said control level indicates that the test substance modulates the glycosylation activity of GALNT6 polypeptide for the substrate; and
(d) selecting the test substance that inhibits the glycosylation activity of GALNT6 polypeptide for the substrate.

5. The method of claim 4, wherein the functional equivalent of the GALNT6 polypeptide comprises a histidine residue of position 271 and/or glutamate residue of position 382 of SEQ ID NO: 29.

6. The method of claim 4, wherein the substrate is a MUC1 polypeptide or functional equivalent thereof.

7. The method of claim 1, wherein the functional equivalent of the MUC1 polypeptide comprises a peptide fragment derived from a variable number tandem repeat (VNTR) domain of the MUC1 polypeptide, wherein the peptide fragment includes one or more serine residues and/or threonine residues.

8. The method of claim 1, wherein the functional equivalent of the MUC1 polypeptide comprises an amino acid sequence of SEQ ID NO: 26(MUC1-a) or SEQ ID NO: 27(MUC1-b).

9. The method of claim 1, wherein the cancer is breast cancer.

10. The method of claim 6, wherein the functional equivalent of the MUC1 polypeptide comprises a peptide fragment derived from a variable number tandem repeat (VNTR) domain of the MUC1 polypeptide, wherein the peptide fragment includes one or more serine residues and/or threonine residues.

11. The method of claim 6, wherein the functional equivalent of the MUC1 polypeptide comprises an amino acid sequence of SEQ ID NO: 26(MUC1-a) or SEQ ID NO: 27(MUC1-b).

12. The method of claim 4, wherein the cancer is breast cancer.
